(19) United States
(12) Patent Application Publication
(10) Pub. No.: US 2026/0092040 A1
(43) Pub. Date: Apr. 2, 2026

(54) SYNTHESIS OF CYCLOPROPANE CARBOXYLIC ACIDS

(71) Applicants: Nikolaos Papaioannou, Lexington, MA (US); Michael Jonathan Fray, Sandwich (GB); Andreas Rennhack, Sandwich (GB); Sarah Jocelyn FINK, Lexington, MA (US); Takeda Pharmaceutical Company Limited, Osaka (JP)

(72) Inventors: Nikolaos Papaioannou, Newton, MA (US); Michael Jonathan Fray, Canterbury, Kent (GB); Andreas Rennhack, Loerrach (GB); Sarah Jocelyn Fink, Arlington, MA (US)

(21) Appl. No.: 19/113,778

(22) PCT Filed: Sep. 20, 2023
(86) PCT No.: PCT/US2023/033264

§ 371 Date: Mar. 20, 2025

Related U.S. Application Data
(60) Provisional application No. 63/408,798, filed on Sep. 21, 2022.

(51) Int. Cl. C07D 239/26; C07D 239/42; C07D 471/04

(52) U.S. Cl. CPC C07D 239/26; C07D 239/42; C07D 471/04

(57) ABSTRACT

The present disclosure provides methods of preparing substituted cyclopropane compounds.

The present disclosure relates to a process for preparing compounds with a substituted cyclopropane with defined stereochemistry and having utility in the preparation of medicinal compounds (in particular plasma kallikrein (pKAL) inhibitors), novel compounds, compounds obtained and obtainable from the process, pharmaceutical formulations comprising any one of the same and use of said medicinal compounds and pharmaceutical formulations in treatment.

### BACKGROUND

The development and execution of the synthesis of hundreds of grams of drug substance is often rate-limiting in the period between nomination of a development candidate and the commencement of Phase I clinical studies. In many cases the original discovery route provides a good starting point, and the process chemist has the task of refining some of the reaction conditions and workups to improve yields and purity, ensuring that the various steps are safe to scale up, and to discover isolation procedures which avoid chromatography if at all possible.

Nowadays the process chemist is faced with syntheses of increasing length and complexity, arising from the use of sophisticated methodologies often requiring difficult-to-handle and expensive catalysts, and drug targets incorporating increasing numbers of sp³ and stereogenic centres.

Cyclopropanes have become popular structural motifs in medicinal chemistry, as they often afford the advantages of restricting conformational freedom at the cost of minimal steric bulk and better stability towards oxidative metabolism compared to lower alkyl or other cycloalkyl substituents.

Certain oral pKAL inhibitors are disclosed in unpublished PCT application number PCT/US2022/020482, and which contain a cyclopropane motif.

The following route shown in Scheme 1 was the discovery chemistry route employed to prepare some of the compounds therein:

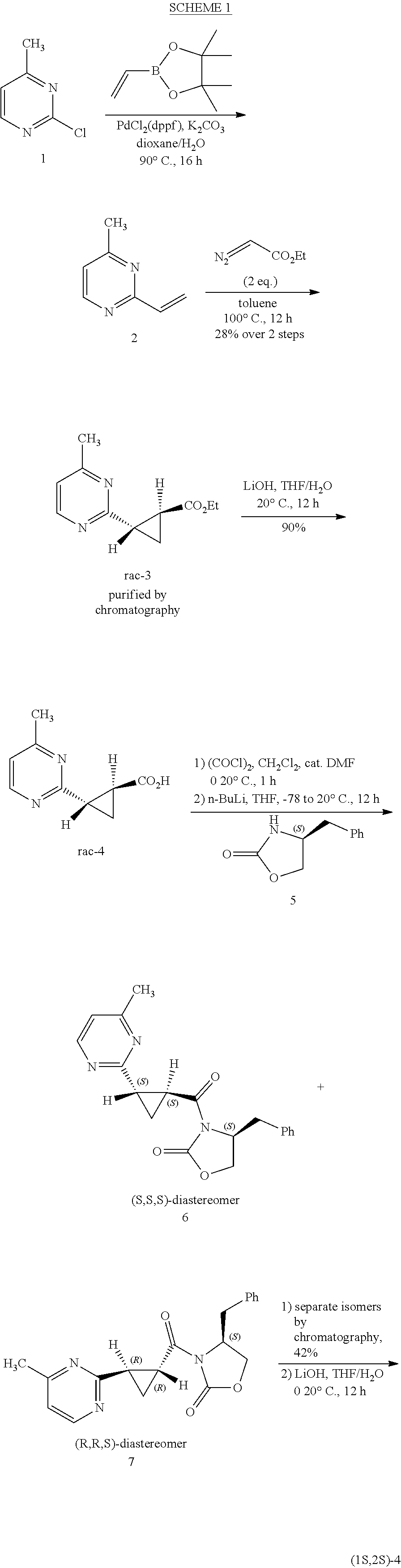

Conversion of compound 1 to compound 2 generated undesirable side reactions and required chromatography to purify them to a suitable level.

When synthetic routes are scaled up for production in a pilot plant, steps such as these in scheme 1 are unsuitable for commercial manufacturing.

A new route of making key intermediates, such as compound 3 is Scheme 1, is required.

The presently disclosed method is safe, scalable and a chromatography-free synthesis, providing efficient resolution via a(S)-1-(1-naphthyl)ethylamine salt. Synthesis of, for example (2RS, 3RS)-3-(4-methylpyrimidin-2-yl) cyclopropane carboxylic acid is provided in 3 steps (58% yield) from 2-chloro-4-methylpyrimidine, and its resolution via recrystallization of the(S)-1-(1-naphthyl)ethylamine or (+)-dehydroabietylamine salts.

The present disclosure provides, among other things, the unexpected development of a palladium-catalysed, conjugate addition of potassium vinyltrifluoroborate to 2-Chloro-4-methylpyrimidine, which forms an intermediate poised for novel cyclopropanation through reaction with a nitrogen ylide derived from t-butyl bromoacetate and DABCO.

### SUMMARY OF THE INVENTION

The invention is summarised in the following paragraphs:

1. A process for the preparation of a trans racemate of formula (I)

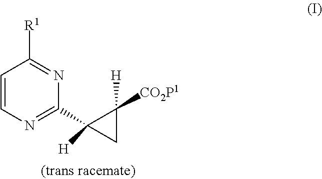

wherein
P¹ is a protecting group (such as tert-butyl)
R¹ is H or optionally substituted C1-C6 alkyl,
said process comprising reacting a compound of formula (II):

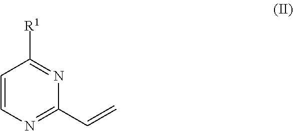

wherein R¹ is defined above for compounds of formula (I)
with a nitrogen ylide, optionally formed in situ, prepared by
reacting an a halo ester, (such as methyl chloroacetate and tert-butyl bromoacetate) with a tertiary amine, (such as DABCO),
to form a quaternary ammonium salt,
followed by treatment with an alkali metal base (such as Cs₂CO₃ or K₂CO₃) and/or an organic base (such as DBU),
in a polar aprotic solvent (such as acetonitrile)
at an elevated temperature, (such as 70-80° C.)
such that the compound of formula (I) is formed (for example with high diastereoselectivity, such as 50:1, over the corresponding cis racemate)

2. A process according to paragraph 1, wherein the a halo ester is tert-butyl bromoacetate.

3. A process according to paragraph 1 or 2, wherein the tertiary amine is DABCO.

4. A process according to any preceding paragraph wherein the alkali metal base is Cs₂CO₃.

5. A process according to any preceding paragraph, wherein the polar aprotic solvent is acetonitrile.

6. A process according to any preceding paragraph wherein the elevated temperature is 70-80° C.

7. A process according to any preceding paragraph, wherein the reaction is performed for 12 to 30 hours, such as 13, 14, 15, 16, 17, 18, 19, 20, 21, 22, 23, 24, 25, 26, 27, 28, 29 or 30, such as 20 hours.

8. A process according to paragraph 1, wherein the steps, reagents and conditions are:

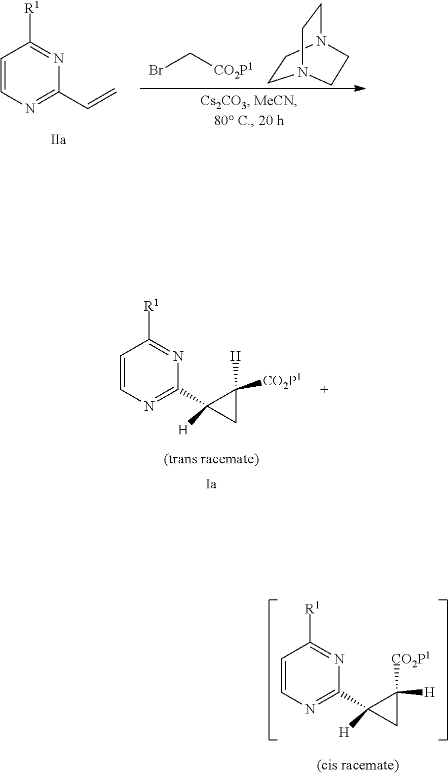

wherein R¹ and P¹ are defined above for compounds of formula (I).

9. A process according to any preceding paragraph 1, wherein a compound of formula (II) is prepared by reacting an intermediate of formula (III)

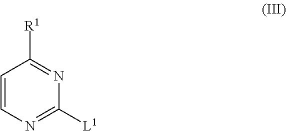

wherein R¹ is defined above for compounds of formula (I) and L¹ represents a leaving group, such as a halogen, in particular Cl
with a vinyltrihaloborate (such as vinyltrifluoroborate, in particular a salt thereof such as potassium).

10. A process according to any preceding paragraph wherein the protecting group P¹ is removed from compounds of formula (I) to liberate the free carboxylic acid of formula

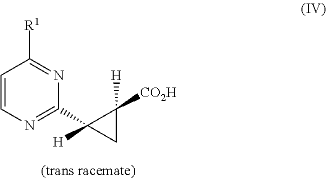

as a trans racemate (for example in high diastereomeric purity, in particular that is essentially free of the cis racemate) by:
a) acidolysis using an organic acid (such as TFA) in a chlorinated solvent (such as DCM); or
b) saponification using an alkali metal hydroxide (such as sodium hydroxide) in an aqueous medium (such THF and water),
   followed by acidification with (for example aqueous hydrochloric acid), to liberate the free acid from the salt so formed.

11. A process for the resolution of a compound of formula (IV) into essentially one or other of its enantiomeric forms, for example to provide a compound of formula (V) (such as absolute as drawn):

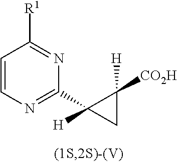

for example wherein the resolution is effected by reaction with an optically pure chiral amine (such as(S)-1-(naphthalen-2-yl) ethanamine or(S)-1-(naphthalen-1-yl) ethanamine), in a suitable solvent (such as ethyl acetate, isopropanol and dimethyl carbonate) to form a mixture of diastereomeric salts from which the preferred diastereomeric salt crystallises and is collected by filtration.

12. A process according to paragraph 11, wherein the free acid is recovered from said salt by treating the latter with an excess of an aqueous solution of an alkali metal hydroxide (such sodium hydroxide or potassium hydroxide),
in the presence of an immiscible organic solvent (such as toluene or MTBE) to remove the organic base, and retain the aqueous solution.

13. A process according to paragraph 12, wherein the aqueous solution is acidified to pH 3-4 with an inorganic acid (such as hydrochloric acid) to precipitate the free acid as a solid which is collected by filtration.

14. A process according to any one of paragraphs 11 to 14, wherein the compound of formula (V) is enantiomerically enriched and in particular having an enantiomeric purity (ee value) of 90% or more, such 91, 92, 93, 94, 95, 96, 96, 97, 98, 99 or 100%, especially 99%.

15. A process according to any preceding paragraph wherein R¹ is C₁₋₃ alkyl, such as methyl, ethyl, propyl or isopropyl, in particular methyl.

16. A process according to any preceding paragraph wherein P¹ is C₁₋₄ alkyl such as t-butyl.

17. A process according to any one of paragraphs 11 to 17, wherein a compound of formula (V) is reacted with an aryl amine of formula (VI):

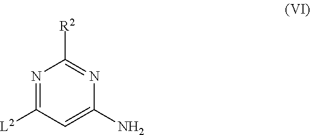

wherein R² is H, or C₁₋₃ alkyl and L², is a leaving group such as a halogen, in particular Cl, to provide a compound of formula (VII):

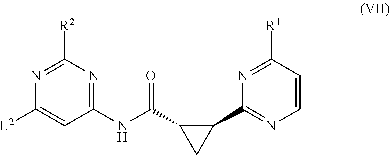

18. A process according to paragraph 17, wherein the compound of formula (VII) is compound (VIIa):

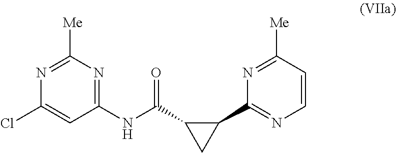 [SMILES: Cc1ccnc([C@H]2C[C@@H]2C(=O)Nc2cc(Cl)nc(C)n2)n1]

19. A process according to paragraph 17, wherein the compound of formula (VII) is compound (VIIb):

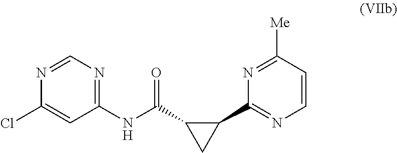 [SMILES: Cc1ccnc([C@H]2C[C@@H]2C(=O)Nc2cc(Cl)ncn2)n1]

20. A process wherein the compound of formula (VII) is reacted with a compound of formula (VIII)

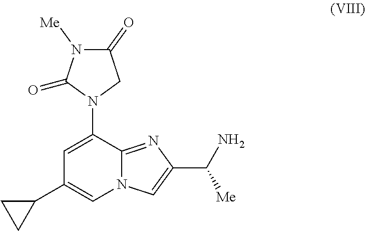 [SMILES: C[C@@H](N)c1cn2cc(C3CC3)cc(N3CC(=O)N(C)C3=O)c2n1]

to provide a compound of formula (IX)

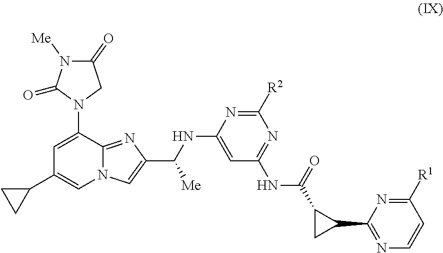

or a pharmaceutically acceptable salt thereof, wherein R¹ and R² are defined above.

21. A process according to paragraph 20, wherein the compound of formula (VII) is compound (VIIa) and the compound of formula (IX) is compound (IXa):

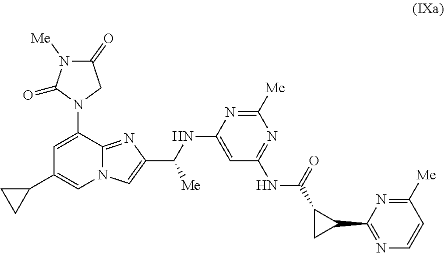

22. A process according to paragraph 20, wherein the compound of formula (VII) is compound (VIIb) and the compound of formula (IX) is compound (IXb):

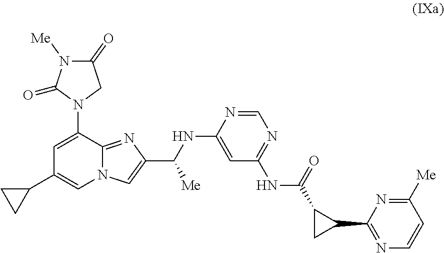

23. A compound obtained or obtainable from any one of the preceding paragraphs.

24. A compound of formula (I), (II), (IV), (V), (VI), (VII), (VIIa) (VIII), (IX), (IXa) or (IXb).

25. A pharmaceutical composition comprising a compound according to paragraph 22 or 23 and an excipient, diluent or carrier.

26. A compound according to paragraph 23 or 24 or a pharmaceutical composition according to paragraph 25, for use in treatment, particularly as a pKAL inhibitor.

### DETAILED DESCRIPTION

### Synthesis of Compounds

Compounds of the disclosure may be synthesized according to the schemes described below and in the ensuing examples. The reagents and conditions described are intended to be exemplary and not limiting. As one of skill in the art would appreciate, various analogs may be prepared by modifying the synthetic reactions such as using different starting materials, different reagents, and different reaction conditions (e.g., temperature, solvent, concentration, catalyst, activator, etc.)

In one aspect, the present disclosure provides methods for the improved synthesis of compounds of formulae (I), (II), (III), (IV), (V), (VII), and (IX) as described above, or salts thereof. It will be appreciated that certain disclosed compounds are novel intermediates of other disclosed compounds, and also are an aspect of the disclosure. In some embodiments of compounds of formulae (I), (II), (III), (IV), (V), (VII), and (IX), R¹ is C₁₋₆ alkyl. Alkyl as used herein refers to straight chain or branched chain alkyl, such as, without limitation, methyl, ethyl, propyl, iso-propyl, butyl, and tert-butyl. In one embodiment alkyl refers to straight chain alkyl, C₁₋₆ alkyl refers to an alkyl group with up to 6 carbons. Similarly, C₁₋₃ alkyl refers to an alkyl group with up to 3 carbons. Optionally substituted alkyl as employed herein refers to wherein 1 or 2 carbon atoms in the alkyl group are replaced with a heteroatom independently selected from N, O or and/or the alkyl group bears 1 to 6 R³ groups as described below:
R³ is oxo, hydroxy, halogen (such as F, Cl), CN, C₁₋₃ alkyl, C₃₋₅ cycloalkyl, —OC₁₋₃ alkyl (such as —OCH₃), —(O)₀₋₁C₁₋₃ alkyl bearing 1 to 6 halogen groups (such as CF₃ or OCHF₂), C₁₋₃ alkyl bearing one or more OR₄ groups, —C(O)C₁₋₃alkylNR⁴R⁵, —SO₂C₁₋₃ alkyl, —SO₂NR⁴R⁵, —NR⁴R³ (such as NH₂);
R⁴ is H or C₁₋₃ alkyl, such as —CH₃; and
R⁵ is H or C₁₋₃ alkyl, such as —CH₃.

### General Synthesis of Certain Cyclopropane Carboxylic Acids

In one aspect, the present disclosure provides methods for the synthesis of compound S1.4, or a salt thereof. In some embodiments, the present disclosure provides methods for the synthesis of compound S1.4 (trans racemate), or a salt thereof. In some embodiments, the present disclosure provides methods for the synthesis of compound S1.4 (optically enriched), or a salt thereof. In some embodiments, such methods are as shown in Scheme A, below:

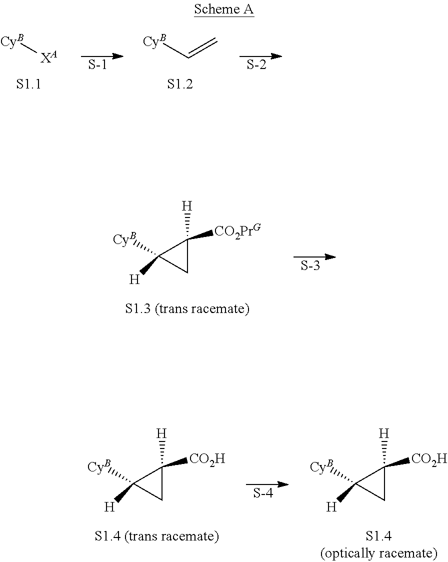

wherein each of Cy⁸, X^{A}, and Pr^{G} are as defined herein.

At step S-1, chemical compound S1.1 undergoes a coupling reaction with a vinyl compound under suitable conditions to provide vinyl compound S1.2. One of ordinary skill in the art will appreciate that a variety of suitable conditions are well known to form an sp²-sp² bond junction between heteroaryls and vinyls. In some embodiments S-1 comprises a Suzuki-Miyaura reaction, conditions for which are known to the skilled artisan.

In some embodiments, step S-1 comprises treating S1.1 with a vinyl boron compound (e.g., vinylboronic acid, pinacol ester or vinyltrifluoroborate) under suitable conditions. In some embodiments, step S-1 comprises treating S1.1 with potassium vinyltrifluoroborate under suitable conditions.

In some embodiments, step S-1 comprises the presence of a suitable base (e.g, pyridine, triethylamine, DBU, tetramethylguanidine, CsF, NaOH, KOH, Na₂CO₃, K₃PO₄, Cs₂CO₃, or K₂CO₃). In some embodiments, step S-1 comprises use of K₂CO₃ as a base.

In some embodiments, step S-1 comprises a suitable pre-catalyst or catalyst (e.g., Pd(OAc)₂·2(2-dicyclohexylphosphino-2′,6′-dimethoxybiphenyl), PdCl₂(PPh₃)₂, or Pd(OAc)₂ dppf). In some embodiments, step S-1 comprises a suitable pre-catalyst such as Pd(OAc)₂. In some embodiments, step S-1 comprises a suitable catalyst ligand such as dppf. In some embodiments, step S-1 comprises the pre-catalyst Pd(OAc), dppf. In some embodiments, step S-1 comprises the pre-catalyst PdCl₂ dppf. In some embodiments, step S-1 comprises the pre-catalyst PdCl₂·(PPh₃)₂.

In some embodiments, step S-1 is carried out at temperatures of about 60° C. to about 120° C. In some embodiments, step S-1 is carried out at temperatures of about 80° C. to about 100° C. In some embodiments, step S-1 is performed at a temperature of about 90° C.

In some embodiments, step S-1 comprises a suitable solvent (e.g., dioxane, water, THF, ethanol, or a combination thereof). In some embodiments, step S-1 solvent is or comprises dioxane. In some embodiments, step S-1 solvent is or comprises water. In some embodiments, step S-1 solvent is or comprises a mixture of dioxane and water.

In some embodiments, step S-1 comprises an inert atmosphere. In some embodiments, step S-1 comprises an atmosphere sufficiently free of oxygen gas. In some embodiments, step S-1 comprises a nitrogen gas atmosphere. In some embodiments, step S-1 comprises an argon gas atmosphere.

In some embodiments, step S-1 comprises a filtration.

In some embodiments, step S-1 comprises a purification. In some embodiments, a purification operation of step S-1 is or comprises a distillation. In some embodiments, step S-1 comprises a vacuum distillation. In some embodiments, step S-1 comprises a purification by vacuum distillation.

In some embodiments, the present disclosure provides a method comprising the steps of.
a) providing compound S1.1 of formula Cy^{B}-X^{A}, wherein Cy^{B} and X^{A} are defined herein; and
b) reacting Cy^{B}-X^{A} under suitable conditions with a vinyl boronate reagent to provide a heteroaromatic vinyl compound of formula S1.2:

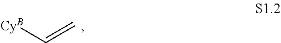

or a salt thereof.

At step S-2, vinyl compound S1.2 undergoes cyclopropanation under suitable conditions to yield trans racemate cyclopropane ester S1.3. Without subscribing to a particular theory, in some embodiments, step S-2 comprises reacting the vinyl group with an ammonium ylide to provide S1.3. In some embodiments, step S-2 comprises forming an ammonium halide salt from an alpha halogen ester (e.g., t-butyl bromoacetate, methyl chloroacetate) and a tertiary amine or nucleophile (e.g., DABCO, quinuclidine, O-methyl quinine, triphenylphosphine, etc) prior to contact with compound of formula S1.2. In some embodiments, step S-2 comprises forming an ammonium halide salt prior to contact with a compound of formula $1.2. In some embodiments, step S-2 comprises forming an ammonium ylide salt prior to contact with a compound of formula S1.2. In some embodiments, step S-2 comprises forming an ammonium ylide salt after contact with a compound of formula S1.2.

In some embodiments, step S-2 comprises a suitable base (e.g., Cs₂CO₃, K₂CO₃, DBU, Ag₂CO₃, potassium tert-butoxide, tetramethylguanidine, triethylamine, etc). In some embodiments, step S-2 comprises a suitable base selected from ground Cs₂CO₃ or ground K₂CO₃. In some embodiments, step S-2 comprises ground Cs₂CO₃.

In some embodiments, step S-2 comprises a suitable solvent (e.g., acetonitrile, dichloromethane, dimethylformamide, tetrahydrofuran, toluene, or dioxane). In some embodiments, step S-2 comprises acetonitrile.

In some embodiments, step S-2 is conducted at a temperature between about 20° C. and about 100° C. In some embodiments, step S-2 is conducted at a temperature between about 70° C. and about 90° C. In some embodiments, step S-2 is carried out at a temperature of about 80° C.

In some embodiments, the present disclosure provides a method comprising the steps of:
a) providing a heteroaromatic vinyl compound of formula S1.2:

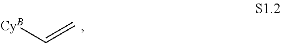

wherein Cy^{B} is defined herein; and
b) reacting the heteroaromatic vinyl compound of formula S1.2 under suitable conditions to provide a cyclopropane ester of formula S1.3:

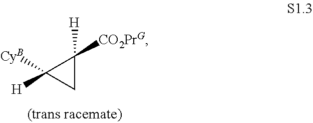

or a salt thereof,
wherein Pr^{G} is a suitable protecting group as defined herein.

At step S-3, cyclopropane ester S1.3 is converted under suitable conditions to trans racemate cyclopropane acid S1.4. At step S-3, the Pr^{G} group of cyclopropane ester S1.3 is removed to provide S1.4. In some embodiments, step S-3 comprises a suitable acid (e.g., trifluoroacetic acid, hydrochloric acid, sulfuric acid, tosic acid, or phosphoric acid). In some embodiments, step S-3 comprises a suitable solvent (e.g., dichloromethane, methanol, ethyl acetate, water, or a combination thereof). In some embodiments, step S-3 comprises a suitable base (e.g., sodium hydroxide). In some embodiments, step S-3 is carried out at temperatures between about 0° C. and about 100° C. In some embodiments, step S-3 is carried out at temperatures between about 5° C. and about 25° C. In some embodiments, step S-3 is carried out at a temperature of about 15° C.

In some embodiments, the present disclosure provides a method comprising the steps of:
a) providing a cyclopropane ester of formula S1.3:

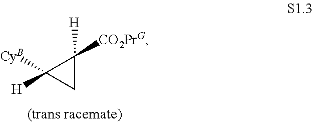

and
b) reacting the cyclopropane ester of formula S1.3 under suitable conditions to provide a carboxylic acid of formula $1.4:

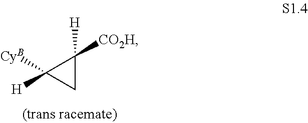

or a salt thereof.

At step S-4, from trans racemate cyclopropane acid S1.4, an enriched enantiomer cyclopropane acid S1.4 (optically enriched) is isolated. In some embodiments, step S-4 comprises chiral chromatography. In some embodiments, step S-4 comprises a chiral resolution (e.g., a chiral salt) under suitable conditions. Chiral salts are known in the art and include by way of non-limiting example crystallization agents such as arylamines and amino alcohols. In some embodiments, step S-4 comprises a chiral salt selected from (+)-dehydroabietylamine, (R)-(+)-1-(2-naphthyl)ethylamine, (S)-(−)-1-(2-naphthyl)ethylamine, or(S)-(−)-1-(1-naphthyl)ethylamine. In some embodiments, step S-4 comprises a chiral resolution agent. In some embodiments, step S-4 comprises the resolution agent(S)-(−)-1-(1-naphthyl)ethylamine. In some embodiments, step S-4 comprises a recrystallization step. In some embodiments, step S-4 comprises one, two, or more recrystallizations. In some embodiments, step S-4 comprises a suitable solvent (e.g., ethyl acetate, isopropyl acetate, acetonitrile, ethanol, dioxane, methanol, dichloromethane, chloroform, isopropanol, tetrahydrofuran, toluene, IMS (industrial methylated spirit; ethanol:methanol 95:5), methanol, tert-butyl methyl ether, water, dioxane, 1,2-dimethoxyethane, 2-methyl tetrahydrofuran, dimethyl carbonate, diethyl carbonate, ethylene carbonate, or a combination thereof). In some embodiments, a suitable (re) crystallization solvent is or comprises dimethyl carbonate. In some embodiments, step S-4 comprises one, two, or three recrystallizations with a co-crystallization agent (e.g., (+)-dehydroabietylamine, (R)-(+)-1-(2-naphthyl)ethylamine, (S)-(−)-1-(2-naphthyl)ethylamine, or(S)-(−)-1-(1-naphthyl)ethylamine). In some embodiments, step S-4 comprises one, two, or three recrystallizations with(S)-(−)-1-(1-naphthyl)ethylamine. In some embodiments, step S-4 comprises one, two, or three recrystallizations with(S)-(−)-1-(1-naphthyl)ethylamine in dimethyl carbonate under reflux conditions. In some embodiments, step S-4 comprises a step of washing a solid with solvent (e.g., dimethyl carbonate). In some embodiments, step S-4 comprises a solvent and compound for recrystallization at a relative concentration of about 0.10 mL/mmol to about 20 mL/mmol. In some embodiments, step S-4 comprises a solvent and compound for recrystallization at a relative concentration of about 0.25 mL/mmol to about 12 mL/mmol. In some embodiments, step S-4 comprises a solvent and compound for recrystallization at a relative concentration of about 0.50 mL/mmol to about 4 mL/mmol. In some embodiments, step S-4 comprises a solvent and compound for recrystallization at a relative concentration of about 0.75 mL/mmol. In some embodiments, step S-4 comprises a solvent and compound for recrystallization at a relative concentration of about 0.25 mL/mmol to about 1.75 mL/mmol. In some embodiments, step S-4 comprises a solvent and compound for recrystallization at a relative concentration of about 0.5 mL/mmol to about 1.25 mL/mmol. In some embodiments, step S-4 comprises a solvent and compound for recrystallization at a relative concentration of about 2 mL/mmol. In some embodiments, step S-4 comprises a solvent and compound for recrystallization at a relative concentration of about 4 mL/mmol. In some embodiments, step S-4 comprises a solvent and compound for recrystallization at a relative concentration of about 12 mL/mmol. In some embodiments, step S-4 comprises a solvent and compound for recrystallization at a relative concentration of about 20 mL/mmol. In some embodiments, step S-4 comprises cooling a refluxing solution of S1.4 and a co-crystallizing agent (e.g., (+)-dehydroabietylamine, (R)-(+)-1-(2-naphthyl)ethylamine, (S)-(−)-1-(2-naphthyl)ethylamine, or(S)-(−)-1-(1-naphthyl)ethylamine) at a rate of about 0.2° C./hour. In some embodiments, step S-4 further comprises treating a recrystallized chiral salt under suitable conditions with a suitable base (e.g., aqueous NaOH) optionally in the presence of a suitable solvent (e.g., dichloromethane or methyl tert-butyl ether) to provide cyclopropane acid S1.4 (optically enriched). In some embodiments, step S-4 comprises treating an extract solution of $1.4 (optically enriched) with a suitable acid (e.g., aqueous HCl). In some embodiments, step S-4 comprises treating an extract solution of S1.4 (optically enriched) with a suitable acid to the endpoint of about pH 3-4 (e.g., about 3.44). In some embodiments, step S-4 comprises collecting precipitated S1.4 (optically enriched) by filtration.

In some embodiments, the present disclosure provides a method comprising the steps of:
a) providing trans racemate cyclopropane carboxylic acid of formula $1.4:

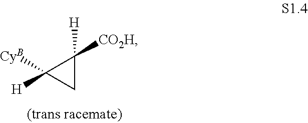

and
b) contacting trans racemate cyclopropane carboxylic acid of formula S1.4 with a chiral chemical environment (e.g., chiral chromatography or a chiral co-crystallization agent such as a chiral amine base), under suitable conditions to provide a cyclopropane carboxylic acid of formula S1.4 (optically enriched):

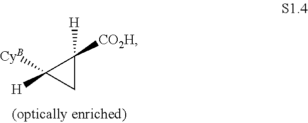

or a salt thereof.

In certain embodiments, each of the aforementioned synthetic steps may be performed sequentially with isolation of each intermediate performed after each step. Alternatively, each of steps S-1, S-2, S-3, and S-4, as depicted in Scheme A above, may be performed in a manner whereby no isolation of one or more intermediates S1.2, S1.3, or S1.4 is performed.

In certain embodiments, all the steps of the aforementioned synthesis may be performed to prepare the desired final product. In other embodiments, two, three, four, five, or more sequential steps may be performed to prepare an intermediate or the desired final product.

It will be appreciated that for optically enriched compounds described herein, it is useful for stereoisomers' (e.g., diastereomers' or enantiomers′) physicochemical characteristics provide for at least one physical means of separation.

It will be appreciated that while a single enantiomer of formula S1.4 (optically enriched) is depicted, the other enantiomer may be isolated and enriched from the resolution as described herein at step S-4, to provide a compound having opposite stereochemistry at each chiral center when a resolution agent, such as (R)-(+)-1-(1-naphthyl)ethylamine is employed.

In some embodiments, Cy^{B} is a 6-membered heteroaryl having 1-3 nitrogens, wherein Cy^{B} is substituted with 0-4-R^{B} groups. In some embodiments, Cy^{B} is a pyrimidinyl group substituted with 0-3-R^{B} groups. In some embodiments, Cy^{B} is a pyridinyl group substituted with 0-4-R^{B} groups. In some embodiments, Cy^{B} is a pyrazinyl group substituted with 0-3-R^{B} groups. In some embodiments, Cy^{B} is a pyridazinyl group substituted with 0-3-R^{B} groups. In some embodiments, Cy^{B} is a 1,2,3,-triazinyl group substituted with 0-2-R^{B} groups. In some embodiments, Cy^{B} is a 1,2,4-triazinyl group substituted with 0-2-R^{B} groups. In some embodiments, Cy^{B} is a 1,3,5-triazinyl group substituted with 0-2-R^{B} groups.

In some embodiments, Cy^{B} is selected from the group consisting of:

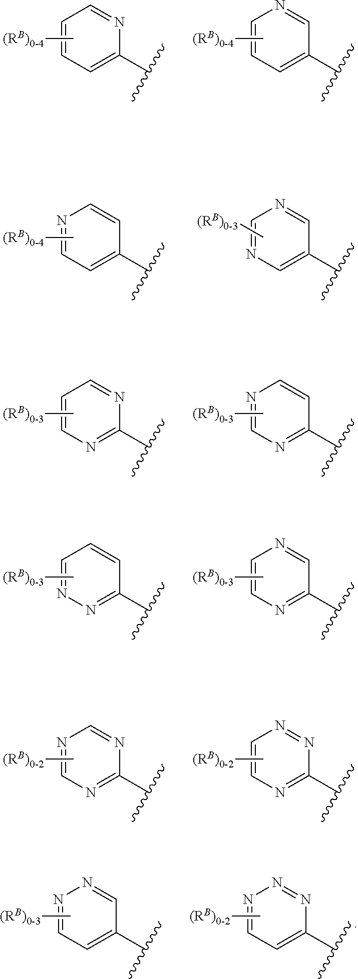

In some embodiments, Cy^{B} is selected from the group consisting of:

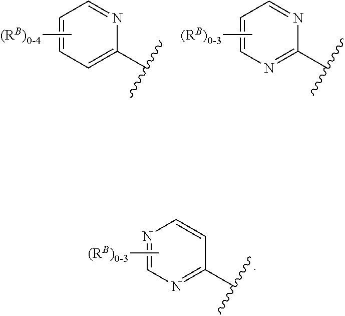

In some embodiments, Cy^{B} is:

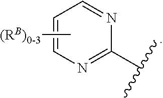

In some embodiments, Cy^{B} is:

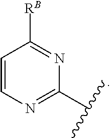

In some embodiments, each R^{B} is independently selected from halogen, —CN, —NO₂, N(R)₂, —N(R)C(O))₂R, —OR, C₁₋₆ aliphatic optionally substituted with halogen, oxo, —OR, or —CN, or a 5-membered heteroaryl having 1-4 heteroatoms independently selected from oxygen, nitrogen, and sulfur, wherein each R is independently hydrogen, C₁₋₆ aliphatic optionally substituted with halogen, oxo, or —CN, a hydroxyl protecting group, or an amino protecting group.

In some embodiments, a single instance of R^{B} is oxo. In some embodiments, a single instance of R^{B} is halogen. In some embodiments, a single instance of R^{B} is fluorine. In some embodiments, a single instance of R^{B} is chlorine. In some embodiments, a single instance of R^{B} is —CN. In some embodiments, a single instance of R^{B} is —NO₂. In some embodiments, a single instance of R^{B} is —N(R)₂. In some embodiments, a single instance of R^{B} is —NHR. In some embodiments, a single instance of R^{B} is —NH₂. In some embodiments, a single instance of R^{B} is —N(R)C(O)₂R. In some embodiments, a single instance of R^{B} is —OR. In some embodiments, a single instance of R^{B} is —OMe. In some embodiments, a single instance of R^{B} is —OCF₃. In some embodiments, a single instance of R^{B} is —OCHF₂.

In some embodiments, a single instance of R^{B} is C₁₋₆ aliphatic optionally substituted with halogen, oxo, —OR, or —CN. In some embodiments, a single instance of R^{B} is C₁₋₆ aliphatic substituted with halogen. In some embodiments, a single instance of R^{B} is methyl. In some embodiments, a single instance of R^{B} is CF₃. In some embodiments, a single instance of R^{B} is CHF₂.

In some embodiments, a single instance of R^{B} is —OR, wherein each R is independently selected from hydrogen or C₁₋₆ aliphatic optionally substituted with halogen.

In some embodiments, a single instance of RE is a 5-membered heteroaryl having 1-4 heteroatoms independently selected from oxygen, nitrogen, and sulfur.

In some embodiments, X^{A} is halogen, triflate, mesylate, or tosylate. In some embodiments, X^{A} is halogen. In some embodiments, X^{A} is chloro, bromo, or iodo. In some embodiments, X^{A} is triflate, mesylate, or tosylate. In some embodiments, X^{A} is chloro. In some embodiments, X^{A} is bromo. In some embodiments, X^{A} is iodo. In some embodiments, X^{A} is triflate.

In some embodiments, Pr^{G} is a suitable carboxylic acid protecting group. In some embodiments, Pr^{G} is methyl. In some embodiments, Pr^{G} is ethyl. In some embodiments, Pr^{G} is tert-butyl. In some embodiments, Pr^{G} is benzyl.

The following numbered embodiments, while non-limiting, are exemplary of certain aspects of the disclosure:

1. A method for preparing an optically enriched compound of formula S1.4:

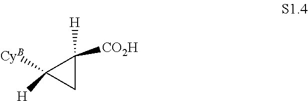

or a salt thereof, wherein:
Cy^{B} is a 6-membered heteroaryl having 1-3 nitrogens, wherein Cy^{B} is substituted with 0-4-R^{B} groups;
each R^{B} is independently selected from halogen, —CN, —NO₂, N(R)₂, —N(R)C(O)₂R, —OR, C₁₋₆ aliphatic optionally substituted with halogen, oxo, —OR, or —CN, or a 5-membered heteroaryl having 1-4 heteroatoms independently selected from oxygen, nitrogen, and sulfur; and
each R is independently hydrogen, C₁₋₆ aliphatic optionally substituted with halogen, oxo, or —CN, a hydroxyl protecting group, or an amino protecting group;
comprising the steps of:
   a) providing trans racemate cyclopropane carboxylic acid of formula S1.4:

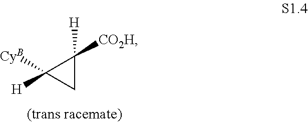

and
b) contacting trans racemate cyclopropane carboxylic acid of formula S1.4 with a chiral chemical environment (e.g., chiral chromatography or a chiral co-crystallization agent such as a chiral amine base), under suitable conditions to provide optically enriched cyclopropane carboxylic acid of formula $1.4:

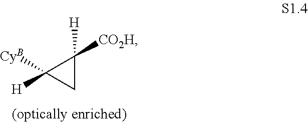

or a salt thereof.

2. The method of embodiment 1, wherein the chiral chemical environment comprises a chiral co-crystallization agent and step b) comprises a recrystallization step.

3. The method of any one of embodiments 1-2, wherein the co-crystallization agent comprises a chiral amine base.

4. The method of embodiment 3, wherein the chiral amine base is selected from (+)-dehydroabietylamine, (R)-(+)-1-(2-naphthyl)ethylamine, (S)-(−)-1-(2-naphthyl)ethylamine, or (S)-(−)-1-(1-naphthyl)ethylamine.

5. The method of embodiment 4, wherein the chiral amine base is(S)-(−)-1-(1-naphthyl)ethylamine.

6. The method of any one of the preceding embodiments, wherein step b) comprises a suitable recrystallization solvent (e.g., ethyl acetate, isopropyl acetate, acetonitrile, ethanol, dioxane, methanol, dichloromethane, chloroform, isopropanol, tetrahydrofuran, toluene, IMS (industrial methylated spirit; ethanol:methanol 95:5), methanol, tert-butyl methyl ether, water, dioxane, 1,2-dimethoxyethane, 2-methyl tetrahydrofuran, dimethyl carbonate, diethyl carbonate, ethylene carbonate, or a combination thereof).

7. The method of embodiment 7, wherein the recrystallization solvent is or comprises dimethyl carbonate.

8. The method of any one of the preceding embodiments, wherein step b) comprises one, two, or three recrystallizations with(S)-(−)-1-(1-naphthyl)ethylamine in dimethyl carbonate under reflux conditions.

9. The method of any one of the preceding embodiments, wherein step b) comprises the step of washing a solid with dimethyl carbonate.

10. The method of any one of the preceding embodiments, wherein step b) comprises a solvent and compound for recrystallization at a relative concentration of about 0.25 mL/mmol to about 12 mL/mmol.

11. The method of embodiment 10, wherein step b) comprises a solvent and compound for recrystallization at a relative concentration of about 0.75 mL/mmol.

12. The method of any one of the preceding embodiments, wherein step b) comprises cooling a refluxing solution of S1.4 and a co-crystallizing agent at a rate of about 0.2° C./hour.

13. The method of any one of the preceding embodiments, comprising treating a recrystallized chiral salt formed in step b) under suitable conditions with a suitable base (e.g., aqueous NaOH) optionally in the presence of a suitable solvent (e.g., dichloromethane or methyl tert-butyl ether) to provide optically enriched cyclopropane acid S1.4.

14. The method of embodiment 13, wherein the base is aqueous NaOH.

15. The method of embodiment 13 or 14, wherein the solvent is dichloromethane.

16. The method of any one of the preceding embodiments, comprising treating an extract solution of optically enriched S1.4 with a suitable acid (e.g., aqueous HCl).

17. The method of embodiment 16, comprising treating an extract solution of optically enriched S1.4 to an endpoint of about pH 3-4 (e.g., about pH 3.44).

18. The method of any one of the preceding embodiments, further comprising the steps of:
c) providing a cyclopropane ester of formula S1.3:

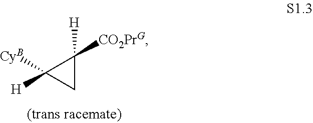

wherein Pr^{G} is a suitable carboxylic acid protecting group, and
d) reacting the cyclopropane ester of formula S1.3 under suitable conditions to provide a carboxylic acid of formula S1.4:

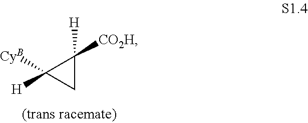

or a salt thereof.

19. The method of claim 18, wherein Pro is t-butyl.

20. The method of embodiment 18 or 19, wherein step d) comprises a suitable acid (e.g., trifluoroacetic acid, hydrochloric acid, sulfuric acid, tosic acid, or phosphoric acid).

21. The method of embodiment 18 or 19, wherein step d) comprises a suitable base (e.g., sodium hydroxide).

22. The method of any one of embodiment 18-21, wherein step d) comprise a suitable solvent (e.g., dichloromethane, methanol, ethyl acetate, water, or a combination thereof).

23. The method of any one of the preceding embodiments, further comprising the steps of: e) providing a heteroaromatic vinyl compound of formula $1.2:

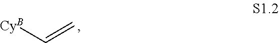

and
f) reacting the heteroaromatic vinyl compound of formula S1.2 under suitable conditions to provide a cyclopropane ester of formula $1.3:

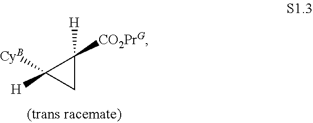

or a salt thereof.

24. The method of embodiment 23, wherein step f) comprises an alpha halogen ester (e.g., t-butyl bromoacetate, methyl chloroactate) and a tertiary amine or nucleophile (e.g., DABCO, quinuclidine, O-methyl quinine, triphenylphosphine, etc).

25. The method of embodiment 24, wherein step f) comprises t-butyl bromoacetate and DABCO.

26. The method of any one of embodiment 23-25, wherein step f) comprises a suitable base (e.g., Cs₂CO₃, K₂CO₃, DBU, Ag₂CO₃, potassium tert-butoxide, tetramethylguanidine, triethylamine, etc).

27. The method of embodiment 26, wherein step f) comprises ground Cs₂CO₃.

28 The method of any one of embodiment 23-27, wherein step f) comprises a suitable solvent (e.g., acetonitrile, dichloromethane, dimethylformamide, tetrahydrofuran, toluene, or dioxane).

29. The method of embodiment 28, wherein step f) comprises acetonitrile.

30. The method of any one of the preceding embodiments, further comprising the steps of:
g) providing compound S1.1 of formula Cy^{B}-X^{A}, wherein X^{A} is halogen, triflate, mesylate, or tosylate; and
h) reacting Cy^{B}-X^{A} under suitable conditions with a vinyl boronate reagent to provide a heteroaromatic vinyl compound of formula S1.2:

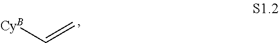

or a salt thereof.

31. The method of embodiment 30, wherein X^{A} is halogen (e.g., chloro).

32. The method of embodiment 30 or 31, wherein the vinyl boronate is potassium vinyltrifluoroborate.

33. The method of any one of embodiments 30-32, wherein step h) comprises the presence of a suitable base (e.g., pyridine, triethylamine, DBU, tetramethylguanidine, CsF, NaOH, KOH, Na₂CO₃, K₃PO₄, Cs₂CO₃, or K₂CO₃).

34. The method of embodiment 33, wherein a suitable base is K₂CO₃.

35. The method of any one of embodiments 30-34, wherein step h) comprises a suitable pre-catalyst or catalyst (e.g., Pd(OAc)₂:2 (2-dicyclohexylphosphino-2′,6′-dimethoxybiphenyl), PdCl₂ (PPh₃)₂, or Pd(OAc)₂·dppf).

36. The method of embodiment 35, wherein step h) comprises Pd(OAc)₂ dppf.

37. The method of any one of embodiments 30-36, wherein step h) comprises a suitable solvent (e.g., dioxane, water, THF, ethanol, or a combination thereof).

38. A method comprising the steps of:
a) providing a cyclopropane ester of formula S1.3:

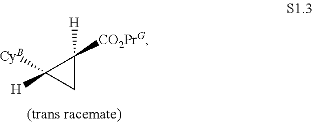

wherein:
Pr^{G} is a suitable carboxylic acid protecting group,
Cy^{B} is a 6-membered heteroaryl having 1-3 nitrogens, wherein Cy^{B} is substituted with 0-4-R^{B} groups;
each R^{B} is independently selected from halogen, —CN, —NO₂, N(R)₂, —N(R)C(O)₂R, —OR, C₁₋₆ aliphatic optionally substituted with halogen, oxo, —OR, or —CN, or a 5-membered heteroaryl having 1-4 heteroatoms independently selected from oxygen, nitrogen, and sulfur; and
each R is independently hydrogen, C₁₋₆ aliphatic optionally substituted with halogen, oxo, or —CN, a hydroxyl protecting group, or an amino protecting group;
and
b) reacting the cyclopropane ester of formula S1.3 under suitable conditions to provide a carboxylic acid of formula S1.4:

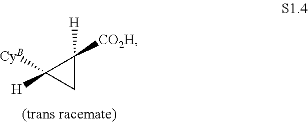

or a salt thereof.

39. A method comprising the steps of:
a) providing a heteroaromatic vinyl compound of formula $1.2:

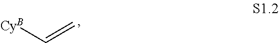

wherein:
Cy^{B} is a 6-membered heteroaryl having 1-3 nitrogens, wherein Cy^{B} is substituted with 0-4-R^{B} groups;
each R^{B} is independently selected from halogen, —CN, —NO₂, N(R)₂, —N(R)C(O)₂R, —OR, C₁₋₆ aliphatic optionally substituted with halogen, oxo, —OR, or —CN, or a 5-membered heteroaryl having 1-4 heteroatoms independently selected from oxygen, nitrogen, and sulfur; and
each R is independently hydrogen, C₁₋₆ aliphatic optionally substituted with halogen, oxo, or —CN, a hydroxyl protecting group, or an amino protecting group;
and
b) reacting the heteroaromatic vinyl compound of formula S1.2 under suitable conditions to provide a cyclopropane ester of formula S1.3:

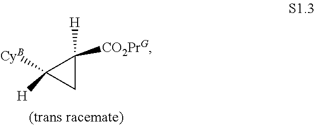

or a salt thereof,
   wherein Pr^{G} is a suitable carboxylic acid protecting group.

40. A method comprising the steps of:
a) providing compound S1.1 of formula Cy^{B}-X^{A},
   wherein:
   Cy^{B} is a 6-membered heteroaryl having 1-3 nitrogens, wherein Cy^{B} is substituted with 0-4-R^{B} groups;
   each R^{B} is independently selected from halogen, —CN, —NO₂, N(R)₂, —N(R)C(O)₂R, —OR, C₁₋₆ aliphatic optionally substituted with halogen, oxo, —OR, or —CN, or a 5-membered heteroaryl having 1-4 heteroatoms independently selected from oxygen, nitrogen, and sulfur; and
   each R is independently hydrogen, C₁₋₆ aliphatic optionally substituted with halogen, oxo, or —CN, a hydroxyl protecting group, or an amino protecting group;
   X^{A} is halogen, triflate, mesylate, or tosylate;
   and
b) reacting Cy^{B}-X^{A} under suitable conditions with a vinyl boronate reagent to provide a heteroaromatic vinyl compound of formula S1.2:

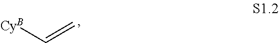

or a salt thereof.

41. The method of any one of the previous embodiments, wherein Cy^{B} is a pyrimidinyl group substituted with 0-3-R^{B} groups.

42. The method of any one of the previous embodiments, wherein Cy^{B} is:

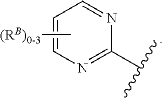

43. The method of any one of the previous embodiments, wherein Cy^{B} is:

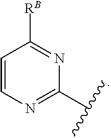

44. The method of any one of the previous embodiments, wherein R^{B} is methyl.

45. The method of any one of the previous embodiments, wherein the compound of formula S1.4 is:

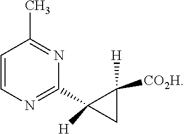

46. The method of any one of the previous embodiments, wherein the compound of formula S1.3 is:

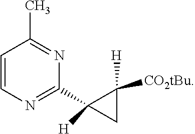 [SMILES: Cc1ccnc([C@H]2C[C@@H]2C(=O)OC(C)(C)C)n1]

47. The method of any one of the previous embodiments, wherein the compound of formula S1.2 is:

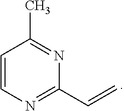

48. The method of any one of the previous embodiments, wherein the compound of formula S1.1 is:

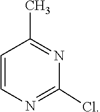

49. The method of any one of the previous embodiments, wherein the optically enriched compound of formula S1.4 has an enantiomeric excess of 90% or more, 95% or more, 96% or more, 97% or more, 98% or more, or 99% or more.

50. A compound of formula S1.4:

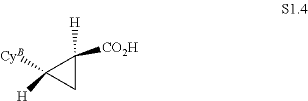

or a salt thereof, wherein:
Cy^{B} is a 6-membered heteroaryl having 1-3 nitrogens, wherein Cy^{B} is substituted with 0-4-R^{B} groups;
each R^{B} is independently selected from halogen, —CN, —NO₂, N(R)₂, —N(R)C(O)₂R, —OR, C₁₋₆ aliphatic optionally substituted with halogen, oxo, —OR, or —CN, or a 5-membered heteroaryl having 1-4 heteroatoms independently selected from oxygen, nitrogen, and sulfur; and
each R is independently hydrogen, C₁₋₆ aliphatic optionally substituted with halogen, oxo, or —CN, a hydroxyl protecting group, or an amino protecting group.

51. The compound of embodiment 50, wherein Cy^{B} is a pyrimidinyl group substituted with 0-3-R^{B} groups.

52. The compound of embodiment 50, wherein Cy^{B} is:

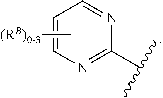

53. The compound of embodiment 50, wherein Cy^{B} is:

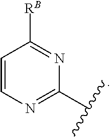

54. The compound of any one of embodiments 50-53, wherein R^{B} is methyl.

55. A compound:

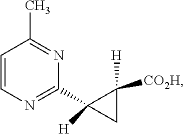

or a salt thereof.

56. A compound:

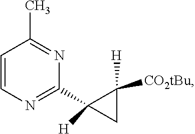 [SMILES: Cc1ccnc([C@H]2C[C@@H]2C(=O)OC(C)(C)C)n1]

or a salt thereof.

57. The compound of any one of embodiments 55 or 56, wherein the compound has an enantiomeric excess of 90% or more, 95% or more, 96% or more, 97% or more, 98% or more, or 99% or more.

### Definitions

Compounds of the disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”. Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The abbreviations used herein have their conventional meaning within the chemical and biological arts. The chemical structures and formulae set forth herein are constructed according to the standard rules of chemical valency known in the chemical arts.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocyclyl,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In some embodiments, aliphatic groups contain 1-4 aliphatic carbon atoms. In some embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocyclyl” or “cycloalkyl”) refers to a monocyclic C₃-C₇ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated,” as used herein, means that a moiety has one or more units of unsaturation.

The terms “halogen” or “halo” includes fluoro, chloro, bromo or iodo, in particular fluoro, chloro or bromo, especially fluoro or chloro.

The term “aryl” refers to monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In some embodiments, an 8-10 membered bicyclic aryl group is an optionally substituted naphthyl ring. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-” refer to groups having 5 to 10 ring atoms, preferably 5, 6, or 9 ring atoms; having 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (or in the case of a bivalent fused heteroarylene ring system, at least one radical or point of attachment is on a heteroaromatic ring). Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted.

As used herein, the terms “heterocyclyl,” “heterocyclic radical,” and “heterocyclic ring” are used interchangeably and refer to a stable 5-to 7-membered monocyclic or 7-10-membered bicyclic heterocyclic moiety that is either saturated or partially unsaturated, and having, in addition to carbon atoms, one or more, preferably one to four, heteroatoms, as defined above. When used in this context in reference to a ring atom, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

By the term “protecting group.” as used herein, it is meant that a particular functional moiety, e.g., O, S, or N, is masked or blocked, permitting, if desired, a reaction to be carried out selectively at another reactive site in a multifunctional compound. Suitable protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. In certain embodiments, a protecting group reacts selectively in good yield to give a protected substrate that is stable to the projected reactions; the protecting group is preferably selectively removable by readily available, preferably non-toxic reagents that do not attack the other functional groups; the protecting group forms a separable derivative (more preferably without the generation of new stereogenic centers), and the protecting group will preferably have a minimum of additional functionality to avoid further sites of reaction. By way of non-limiting example, hydroxyl protecting groups include methyl, methoxylmethyl (MOM), methylthiomethyl (MTM), 1-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, i-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri (p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2-or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-1-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-1-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino) acetamide, 3-(p-hydroxyphenyl) propanamide, 3-(o-nitrophenyl) propanamide, 2-methyl-2-(o)-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N-(N′,N′-dimethylaminomethylene)amine, N,N′-isopropylidenediamine, N-p-nitrobenzylideneamine, N-salicylideneamine, N-5-chlorosalicylidencamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium-or tungsten) carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present disclosure is not intended to be limited to these protecting groups; rather, a variety of additional equivalent protecting groups can be readily identified using the above criteria and utilized in the method of the present disclosure. Additionally, a variety of protecting groups are described by Greene and Wuts (supra).

In certain embodiments, the neutral forms of the compounds are regenerated by contacting the salt with a base or acid and isolating the parent compound in the conventional manner. In some embodiments, the parent form of the compound differs from the various salt forms in certain physical properties, such as solubility in polar solvents and non-polar solvents.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C-or ¹⁴C-enriched carbon are within the scope of this disclosure. Such compounds are useful, for example, as analytical tools, as probes in biological assays, or as therapeutic agents in accordance with the present disclosure. In some embodiments, compounds of the present disclosure are provided as a single enantiomer or single diastereoisomer. Single enantiomer refers to an enantiomeric excess of 80% or more, such as 81, 82, 83, 84, 85, 86, 87, 88, 89, 90, 91, 92, 93, 94, 95, 96, 97, 98 or 99%. Single diastereoisomer excess refers to an excess of 80% or more, for example 81, 82, 83, 84, 85, 86, 87, 88, 89, 90, 91, 92, 93, 94, 95, 96, 97, 98 or 99%. Where a particular enantiomer is preferred, it may, in some embodiments be provided substantially free of the opposite enantiomer, and may also be referred to as “optically enriched.” “Optically enriched,” as used herein, means that the compound is made up of a significantly greater proportion of one enantiomer. In certain embodiments the compound is made up of at least about 80% by weight of a preferred enantiomer. In other embodiments the compound is made up of at least about 90%, 91%, 92%, 93%, 94%, 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972).

The term “oxo,” as used herein, means an oxygen that is double bonded to a carbon atom, thereby forming a carbonyl.

The symbol “w”, except when used as a bond to depict unknown or mixed stereochemistry, denotes the point of attachment of a chemical moiety to the remainder of a molecule or chemical formula.

The articles “a” and “an” are used herein to refer to one or to more than one (i.e., to at least one) of the grammatical object of the article. By way of example, “an element” means one element or more than one element.

The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Examples of pharmaceutically acceptable salts include without limitation, acid addition salts of strong mineral acids such as HCl and HBr salts and addition salts of strong organic acids such as a methansulfonic acid salt.

In the context of this specification “comprising” is to be interpreted as “including”. Embodiments of the invention comprising certain features/elements are also intended to extend to alternative embodiments “consisting” or “consisting essentially” of the relevant elements/features.

Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

Subject headings herein are employed to divide the document into sections and are not intended to be used to construe the meaning of the disclosure provided herein.

The background section contains technical information relating to the invention and may be employed as basis for amendment.

The present invention is further described by way of illustration only in the following examples.

### Exemplification

As depicted in the Examples below, in certain exemplary embodiments, compounds are prepared according to the following general procedures. It will be appreciated that, although the general methods depict the synthesis of certain compounds of the present disclosure, the following general methods, and other methods know to one of ordinary skill in the art, can be applied to all compounds and subclasses and species of each of these compounds as described herein. General Considerations. All non-aqueous reactions were carried out in oven-or heatgun-dried glassware under an inert atmosphere of nitrogen and employing standard techniques for handling air-sensitive materials. Cesium carbonate was obtained from Fluorochem (cat. 050215), ground to a fine powder (pestle and mortar), passed through a stainless steel sieve (150 μm) under a nitrogen atmosphere, dried (120° C., 80 mbar, 24-48 h) and sieved again prior to use. (+)-Dihydroabietylamine was obtained from TCI (cat. D1588, 90% purity) and used as received, adjusting for purity. Acetonitrile (HPLC grade) was dried over 4 Å molecular sieves for 24 h. All other chemicals and solvents (HPLC grade or anhydrous as required) were purchased from commercial sources and used as received. Dicalite is a brand of diatomaceous earth (Kieselguhr) supplied by Dicalite Minerals Corp. NMR spectra were measured with a Jeol 400YH spectrometer operating at 396 MHz (¹H), and 100 MHz (¹³C).

Data were processed using Jeol Delta software and shifts are quoted in ppm Proton and carbon chemical shifts are referenced to residual protonated solvent. Resonances are described as s (singlet), d (doublet), t (triplet), q (quartet), m (multiplet), br (broad), dd (double doublet), and so on. Coupling constants (J) are given in Hertz (Hz) and are accurate to the nearest 0.3 Hz. Solvents used for samples are specified in the specific experimental procedures for each compound. UPLCMS data were obtained using a Waters Acquity system employing either of the two following methods. Method A (Waters Acquity BEH C18, 1.7 μm, 2.1×50 mm, 0.4 mL/min, A: H₂O:MeCN=95:5+0.1% 28% NH₃ aq. B: H₂O:MeCN=5:95+0.1% 28% NH₃ aq; Gradient: 0-0.2 min 100% A, 0.2×3.5 min ramp to 100% B, 3.5-4.5 min 100% B). Method B (Waters Acquity CSH C₁₈, 1.7 μm, 2.1×50 mm, 0.4 mL/min, A: H₂O:MeCN=95:5+0.1% HCOOH, B. H₂O:MeCN=5.95+0.1% HCOOH, Gradient: 0-0.2 min 100% A, 0.2-3.5 min ramp to 100% B, 3.5-4.5 min 100% B. Purity was calculated from the relative peak areas (total absorbance from 215 to 350 nm). Chiral SFC was performed on a Shimadzu Nexera SFC system using a Phenomenex Lux Cellulose 2 column (4.6×250 mm, 2 mL/min, 40° C.) and a gradient of scCO₂ [A]: MeOH (+0.1% diethylamine) [B], Gradient: 0-1 min 15% B, 1-9 min ramp to 40% B. The enantiomeric excess was calculated from the respective peak areas at 254 nm. High resolution mass spectra were recorded using an Agilent 6530 accurate mass quadrupole time-of-flight (Q-TOF) LC/MS system operating in positive ionization mode. The m/z values are reported in Daltons. High resolution values were calculated to four decimal places from the molecular formula, all found values being within a tolerance of 5 ppm. Combustion analysis data were obtained by OEA Laboratories Ltd., Callington, Cornwall, U.K. and are the mean of duplicate determinations. Melting points were obtained using a Buchi B545 melting point apparatus in open tubes and are uncorrected. TLC was performed on silica gel 60 F254 glass-backed plates (from Merck KGaA, Darmstadt, Germany) and visualized using ultraviolet light or 0.5% aq. KMnO₄. Rf values are reported with the solvent system used. Flash chromatography refers to column chromatography on silica gel (Silicycle, 40-63 μm, pore diameter 60 Å) using glass columns. Preparative reversed phase column chromatography was performed using a Biotage Isolera system with Biotage Sfåar C₁₈ cartridges (30 g, 30 μm, 25 mL/min) using gradient (A. H₂O+0.1% HCOOH, B: MeCN+0.1% HCOOH, A: B=95:5 (3 column volumes), then ramp to 75:25 over 5 column volumes and hold for 5 column volumes.

DrySyn refers to the range of metal reaction heating blocks available from Asynt (Isleham, nr. Ely, Cambridgeshire, UK). For controlled cooling of recrystallizations, a PolyBLOCK Parallel Chemistry Reaction Block from H.E.L. Group Ltd., was used.

### Example 1

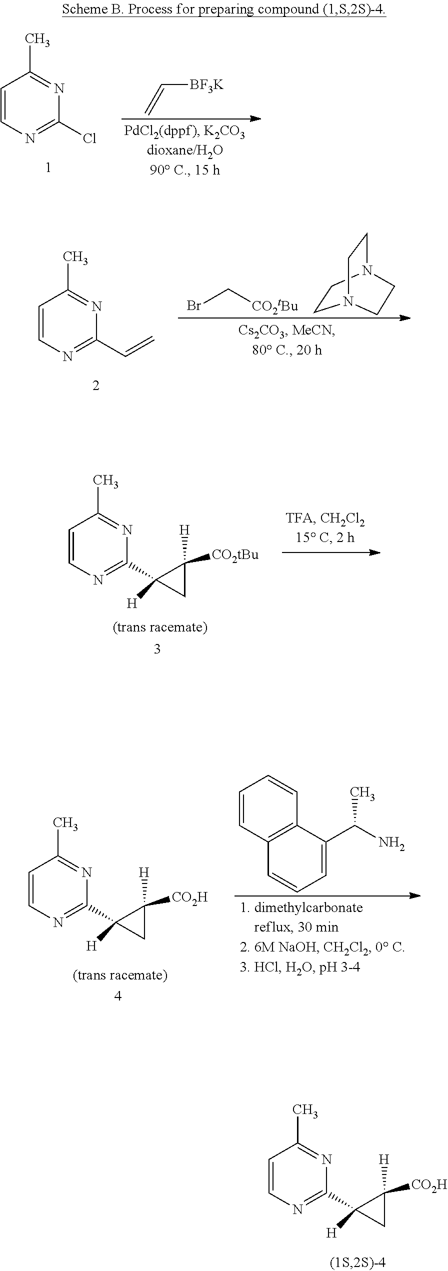

### Step 1. Preparation of 4-methyl-2-vinylpyridine (2)

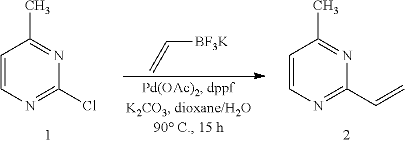

A 5 L 3-neck flask equipped with a mechanical stirrer, nitrogen inlet (via long needle) and outlet was charged with water (500 mL). K₂CO₃ (538 g, 3.90 mol) was added gradually with vigorous stirring until a clear solution was obtained (ca. 15 min). To this was added 1,4-dioxane (2.5 L, BHT stabilized) and the mixture was sparged with nitrogen for 3 h at room temperature. 2-Chloro-4-methylpyrimidine (250 g, 1.95 mol) and potassium vinyltrifluoroborate (302 g, 95% purity, 2.14 mol) were added and sparging was continued for another 30 min. Pd(OAc)₂ (4.36 g, 19.4 mmol) and dppf (10.8 g, 19.4 mmol) were added. A reflux condenser (with nitrogen inlet) and a thermocouple were fitted, and the mixture was heated to 90° C. (internal temperature) for 15 h using an isomantle. The reaction vessel was placed in an ice bath to cool. The mixture was diluted with Et₂O (2 L) and stirred for 30 min. The mixture was filtered through a pad of Dicalite (0.5 Kg). The filtrate was concentrated on a rotary evaporator (42° C. bath temperature, final pressure 90 mbar) to give a dark brown liquid (ca. 1 Kg). Analysis by ¹H NMR showed a mixture of product and dioxane (20:80, w/w). The mixture was distilled under vacuum using a 15 cm Vigreux column (DrySyn at 65° C., 110-130 mbar) to remove the remaining dioxane (bp 46-38° C.). The mixture was then transferred to a 500 mL round bottom flask and distilled further (Drysyn at 92° C., 30-37 mbar) to obtain 4-methyl-2-vinylpyrimidine (203 g), as a colourless liquid. Analysis by ¹H NMR showed the product to contain 11% w/w dioxane; thus, the yield was calculated to be 181 g (77%). The product was stored at −20° C. until needed. UPLCMS (Waters Acquity BEH C18, 1.7 μm, 2.1×50 mm, 0.4 mL/min, A: H₂O:MeCN=95:5+0.1% 28% NH₃ aq, B: H₂O:MeCN=5:95+0.1% 28% NH₃ aq; Gradient: 0-0.2 min 100% A, 0.2-3.5 min ramp to 100% B, 3.5-4.5 min 100% B), RT=1.92 min; [M+H]⁺ 121, purity 99.8%, ¹H NMR (396 MHz, CDCl₃, ppm) δ 8.54 (d, J 5 Hz, 1H), 7.00 (d, J 5 Hz, 1H), 6.85 (dd, J 18, 10 Hz, 1H), 6.61 (dd, J 18, 2 Hz, 1H), 5.71 (dd, J 10, 2 Hz, 1H), 2.52 (s, 3H).

### Step 2. Preparation of (rac-trans)-tert-butyl-2-(4-methylpyrimidin-2-yl) cyclopropane-1-carboxylate (3)

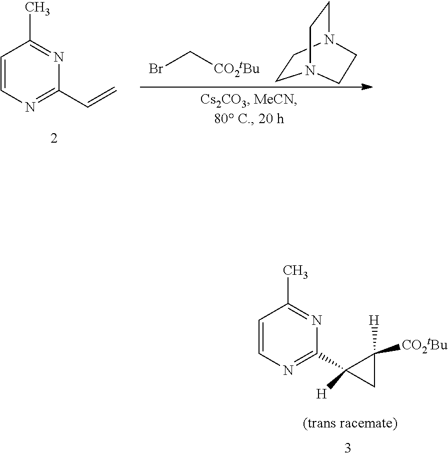

A dried 5 L 3-necked flask was equipped with mechanical stirrer, a temperature probe linked to a heating mantle, and a nitrogen inlet. DABCO (140 g, 1.25 mol) was charged to the flask via a solid addition funnel under nitrogen. Anhydrous MeCN (1.55 L, 3.5 ppm H₂O by KF titration) was added and the mixture was stirred for 5 min to obtain a colourless solution. Tert-butyl bromoacetate (245 g, 1.25 mol) was charged to a pressure-equalizing dropping funnel and washed in with anhydrous MeCN (100 mL). The tert-butyl bromoacetate solution was added via dropping funnel over 30 min, during which time the temperature rose to 44° C. The colorless solution was stirred for 60 min at 23° C., and complete conversion to the ammonium ylide was confirmed by ¹H NMR. 4-Methyl-2-vinylpyrimidine (113 g, 89% purity, 836 mmol, remainder 1,4-dioxane) was added and washed into the flask with anhydrous MeCN (20 mL). Dried, powdered Cs₂CO₃ (<150 μm particle size, 409 g, 1.25 mol) was added to the reaction mixture with stirring in one portion through a solid addition funnel to give a fine suspension. The resulting mixture was heated to 80° C. (internal temperature) over 30 min and maintained for 20 h. The reaction was cooled to 50° C. The mixture was diluted with EtOAc (1.2 L) and filtered through a 3 cm pad of Dicalite filter aid, washing the filter cake with EtOAc (1 L). The filtrate was concentrated under reduced pressure to give a dark brown oil containing some solid, which was cooled in an ice bath and treated with EtOAc (1 L) and aq. HCl (2 M, 500 mL in portions) to give pH 4. The organic layer was separated, and the aqueous layer was extracted with EtOAc (2×500 mL). The combined extracts were washed with sodium phosphate buffer (0.5 M, pH 7, 2×500 mL), dried (Na₂SO₄), filtered and concentrated under reduced pressure to give crude trans-rac-tert-butyl-2-(4-methylpyrimidin-2-yl) cyclopropane-1-carboxylate (190 g, estimated yield 86%) as a brown oil, which was directly used in the next step. A small sample was purified for analysis by flash column chromatography (heptane: EtOAc=7:1-3:1) to obtain a colorless oil. UPLCMS (Waters Acquity BEH C18, 1.7 μm, 2.1×50 mm, 0.4 mL/min, A: H₂O:MeCN=95:5+0.1% 28% NH₃ aq, B: H₂O:MeCN=5:95-0.1% 28% NH₃ aq; Gradient: 0-0.2 min 100% A, 0.2-3.5 min ramp to 100% B, 3.5-4.5 min 100% B) RT=3.23 min; m/z [M+H]⁺ 235 and [M+H-isobutylene]⁺ 179, purity 100%. ¹H NMR (396 MHz, CDCl₃) δ 8.40 (d, J 5 Hz, 1H), 6.94 (d, J 5 Hz, 1H), 2.70 (ddd, J 4, 6.8, 8 Hz, 1H), 2.45 (s, 3H), 2.21 (ddd, J 4, 5.8, 8.2 Hz, 1H), 1.60-1.54 (m, 2H), 1.45 (s, 9H).

### Step 3. Preparation of (rac-trans)-2-(4-methylpyrimidin-2-yl) cyclopropane-1-carboxylic acid (rac-4)

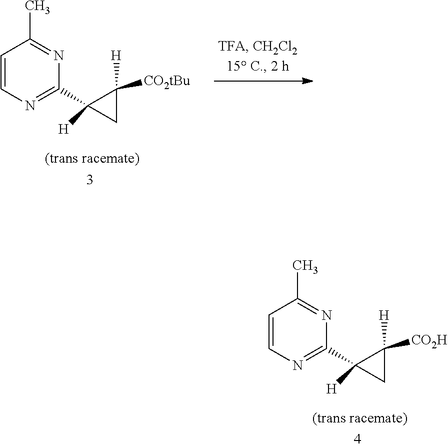 [SMILES: Cc1ccnc([C@H]2C[C@@H]2C(=O)OC(C)(C)C)n1 ; Cc1ccnc([C@H]2C[C@@H]2C(=O)O)n1]

Trifluoroacetic acid (840 mL) was charged to a 3 L round-bottomed flask equipped with a magnetic stir-bar under nitrogen and cooled in an ice-water bath to an internal temperature of 10° C. A solution of (rac-trans)-tert-butyl-2-(4-methylpyrimidin-2-yl) cyclopropane-1-carboxylate (crude, 190 g) in DCM (290 mL) was added via dropping funnel over 18 min during which time the temperature rose to 19° C. Once the addition was finished, the mixture was stirred for another 10 min. The ice bath was removed, and the brown solution was stirred at room temperature for another 60 min. The solution was concentrated under reduced pressure and the residue was co-evaporated from toluene (3×500 mL) to give a viscous brown oil (ca. 420 g). The residue was cooled in an ice bath while aq. NaOH (2 M, ca 1020 mL) was added gradually (to give pH 4). The mixture was stirred in the ice bath for 60 min, and the solid was collected by filtration, washed with water (2×250 mL), and dried by suction overnight. The title compound was obtained as off-white solid (112 g, 75% over 2 steps). Recrystallization from EtOAc gave colorless needles, mp 172.5-173° C. UPLCMS (Waters Acquity CSH C₁₈, 1.7 μm, 2.1×50 mm, 0.4 mL/min, A: H₂O:MeCN=95:5+0.1% HCOOH, B: H₂O:MeCN=5:95+0.1% HCOOH, Gradient: 0-0.2 min 100% A, 0.2-3.5 min ramp to 100% B, 3.5-4.5 min 100% B) RT=1.94 min; [M+H]⁺ 179, purity: 98.5%. Chiral SFC: RT=4.55 min (S,S) and 5.42 min (R,R)=50:50. ¹H NMR (396 MHz, CD₃OD, ppm) δ 8.47 (d, J 5.2 Hz, 1H), 7.17 (d, J 5.2 Hz, 1H), 2.67 (ddd, J 3.8, 6, 9 Hz, 1H), 2.47 (s, 3H), 2.20 (ddd, J 3.8, 5.6, 8.7 Hz, 1H), 1.65-1.57 (m, 2H).

### Step 4. Preparation of (1S,2S)-2-(4-methylpyrimidin-2-yl) cyclopropane-1-carboxylic acid (S,S-4)

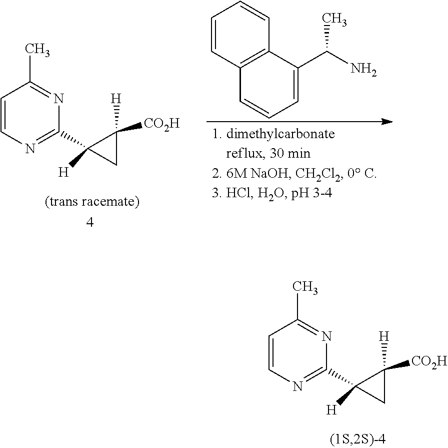

(S)-(−)-1-(1-Naphthyl)ethylamine (45.40 g, 266 mmol) was dissolved in dimethyl carbonate (100 mL) and (rac-trans)-2-(4-methylpyrimidin-2-yl) cyclopropane-1-carboxylic acid (47.26 g, 266 mmol) was added with the aid of more dimethyl carbonate (100 mL). The mixture became warm and almost all the solid dissolved before a solid started to precipitate. The resulting thick slurry was heated under reflux at which point most of the solid was in solution. After 30 min, the solution was allowed to cool to room temperature with stirring overnight. The solid was filtered, washed with dimethyl carbonate (100 mL and 80 mL) and dried to obtain a colorless solid (40.86 g), which was analyzed by chiral SFC chromatography. The ratio of diastereomeric salts was found to be (S,S):(R,R)=94.6:5.4 (89.2% ee). The solid was then suspended in dimethyl carbonate (400 mL) and heated under reflux for 40 min. The suspension was allowed to cool to room temperature, and the solid was filtered and washed with dimethyl carbonate (2× 80 mL) to give a colorless solid (37.79 g), which was analyzed by chiral SFC chromatography. The ratio of diastereomeric salts was found to be (S,S):(R,R)=98.7:1.3 (97.4% ee). This batch (37.79 g) was combined with another batch (2.64 g, 98% de) and triturated with hot dimethyl carbonate (200 mL), as above, for 40 min. The suspension was allowed to cool to room temperature, and the solid was filtered and washed with dimethyl carbonate (2×50 mL) to give a colorless solid (38.95 g, 82% of theory), which was analyzed by chiral SFC chromatography. The ratio of diastereomeric salts was found to be (S,S):(R,R)=99.4:0.6 (98.8% ee). Needles (from dimethyl carbonate) mp 164-165° C. ¹H NMR (396 MHz, CD₃SOCD₃) δ 8.49 (d, J 5 Hz, 1H), 8.17 (d, J 8 Hz, 1H), 7.93 (d, J 9.6 Hz, 1H), 7.81 (d, J 8.4 Hz, 1H), 7.73 (d, J 8.8 Hz, 1H), 7.56-7.49 (m, 3H), 7.17 (d, J 5 Hz, 1H), 4.93 (q, J 6.4 Hz, 1H), 2.48-2.43 (m, 1H), 2.40 (s, 3H), 1.99-1.95 (m, 1H), 1.43 (d, J 6.4 Hz, 3H), 1.44-1.38 (m, 2H).

The(S)-(−)-(1-naphthyl)ethylamine (1S,2S)-4 salt (64.43 g, 185 mmol) was suspended in DCM (200 mL) and cooled in a cold water bath. Aq. NaOH (6 M, 46.1 mL, 277 mmol) was added in portions over 10 min. The mixture was stirred for 10 min to give a clean, two-phase mixture with a faint brown tinge. The mixture was transferred to a separating funnel with water (2×10 mL). The organic layer was separated, and the aqueous layer was washed with dichloromethane (4×50 mL). The aqueous layer was cooled in an ice-water bath and treated with conc. HCl to give pH 3-4. The precipitated solid was collected by filtration and washed with water (3×20 mL) to give (1S,2S)-2-(4-methylpyrimidin-2-yl) cyclopropane-1-carboxylic acid (31.38 g, 95%). mp 192-194° C. UPLCMS (same method as previous step) RT=1.86 min; [M+H]⁺179, purity: 100%. Chiral SFC (S,S):(R,R)=99.76:0.24 (99.5% ee). ¹H NMR (396 MHz, CD₃OD, ppm) δ 8.46 (d, J 5.6 Hz, 1H), 7.17 (d, J 5.6 Hz, 1H), 2.67 (ddd, J 3.7, 6.0, 9.5 Hz, 1H), 2.47 (s, 3H), 2.19 (ddd, J 3.9, 5.5, 8.4 Hz, 1H), 1.64-1.57 (m, 2H).

### Example 2

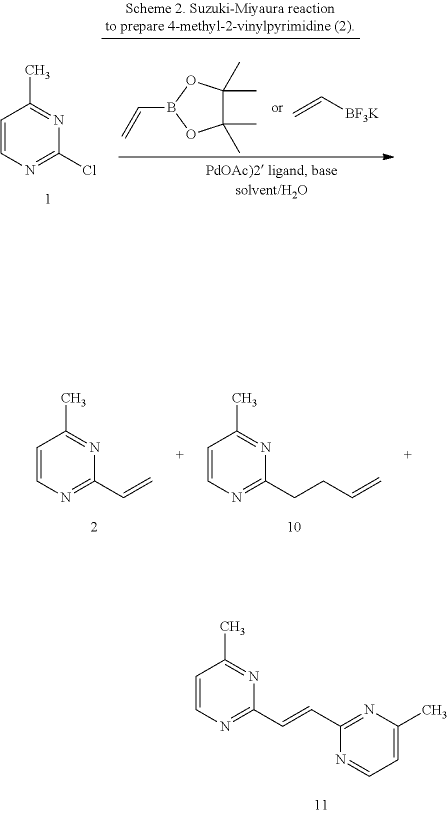

Step S-1 was also conducted according to the parameters of Table 1, with each reaction performed in a manner analogous to the details as described in Example 1 Step 1.

TABLE 1
Suzuki-Miyaura reaction optimization for preparation of 4-methyl-2-vinylpyrimidine (2).

| | Nuc | Base | Catalyst^{a} | Solvents | | Time | Yield^{c} | 2 | 1 | 10 | 11 |
|---|---|---|---|---|---|---|---|---|---|---|---|
| Entry | (cq) | (cq) | (cq) | (ratio) | Temp. | (h) | 2 | (%)^{d} | (%)^{d} | (%)^{d} | (%)^{e} |
| 1 | CH₂═CHBPin | K₃PO₄ | Pd(OAc)₂•2SPhos | Dioxane^{f} | 100° C. | 0.83 | 48% | 97 | 0 | 3 | <1 |
| | (1.5) | (3) | (0.04) | | | | | | | | |
| 2 | CH₂═CHBPin | K₃PO₄ | Pd(OAc)₂•2SPhos | Dioxane^{g} | 90° C. | 18 | ND | 82 | 0 | 10 | <1 |
| | (1.5) | (3) | (0.03) | | | | | | | | |
| 3 | CH₂═CHBF₃K | K₃PO₄ | Pd(OAc)₂•2SPhos | Dioxane^{h} | 90° C. | 3 | 50% | 66 | 6 | 25 | 4 |
| | (1.25) | (3) | (0.03) | | | | | | | | |
| 4 | CH₂═CHBF₃K | K₃PO₄ | Pd(OAc)₂•2SPhos | Dioxane/H₂O | 90° C. | 4.3 | 50% | 56 | 13 | 31 | <1 |
| | (1.25) | (3) | (0.03) | (75:25) | | | | | | | |
| 5 | CH₂═CHBF₃K | K₃PO₄ | PdCl₂(PPh₃)₂ | THF/H₂O | Reflux | 28 | ND | 78 | 22 | 0 | <1 |
| | (1.5) | (3) | (0.05) | (91:9) | | | | | | | |
| 6 | CH₂═CHBF₃K | Cs₂CO₃ | PdCl₂(PPb₃)₂ | THF/H₂O | Reflux | 15 | 48% | 93 | 0 | 7 | <1 |
| | (1.5) | (2.5) | (0.05) | (14:86) | | | | | | | |
| 7 | CH₂═CHBF₃K | K₂CO₃ | Pd(OAc)₂•dppf | Dioxane/H₂O | 90° C. | 16 | 55% | 96 | 0 | 0 | 3 |
| | (1.05) | (2) | (0.03) | (90:10) | | | | | | | |
| 8 | CH₂═CHBF₃K | K₂CO₃ | Pd(OAc)₂•dppf | Dioxane/H₂O | 90° C. | 15 | 77% | 98.5 | 0 | 0 | 1.5 |
| | (1.1) | (2) | (0.01) | (83:17) | | | | | | | |
| 9 | CH₂═CHBF₃K | K₂CO₃ | Pd(OAc)₂•dppf | THF/H₂O | Reflux | 39 | ND | 90 | 10 | 0 | <1 |
| | (1.1) | (2) | (0.01) | (86:14) | | | | | | | |

Table 1.
^{a}SPhos = 2-dicyclohexylphosphino-2′,6′-dimethoxybiphenyl;
^{b}ND = not determined;
^{c}yield of isolated 3, after chromatography or distillation, adjusted for residual solvent;
^{d}calculated from peak area in LC trace, adjusted by correlation with ¹H NMR spectrum;
^{e}calculated from peak area in LC trace;
^{f}+H₂O (7 equiv.);
^{g}+H₂O (5 equiv.);
^{h}+H₂O (2.3 equiv.).

To counter the problem of the product volatility, vinylboronic acid pinacol ester was replaced by potassium vinyltrifluoroborate (entries 3 & 4, under ‘low water’ and conventional ‘wet’ conditions), which gave a similar yield of 3, but even more of impurity 10. A Heck reaction of 3 with 2 readily explains the formation of 11. However, 10 would seem to be generated from a conjugate addition of a vinyl boron species on vinylpyrimidine 3 coordinated to palladium, followed by proto-depalladation rather than the expected β-hydride elimination to give diene 12 (Scheme 3).

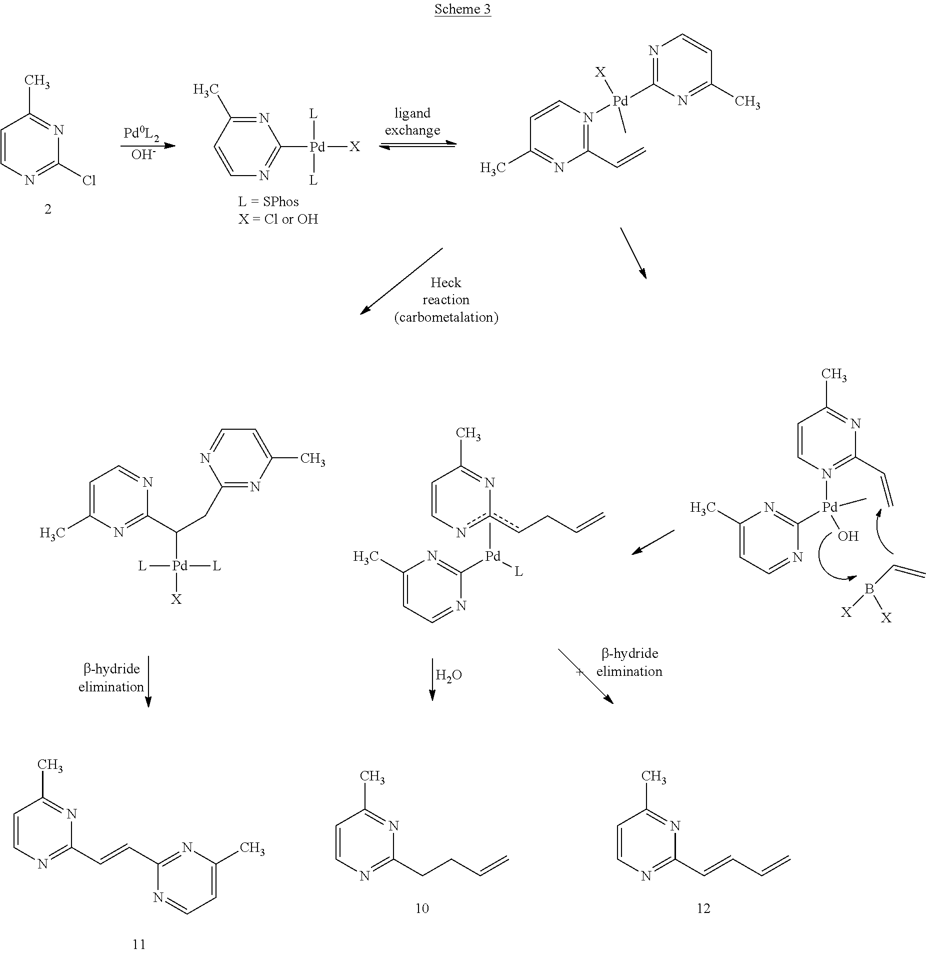

Two trial reactions with bis(triphenylphosphine) palladium chloride in THF under conventional and ‘high water’ conditions with potassium phosphate or cesium carbonate as base (entries 5 & 6) did not improve the yield but seemed to suppress the formation of 10. The best results were obtained with palladium acetate and dppf as catalyst (entries 7 & 8) in dioxane/water with potassium carbonate as base. The number of equivalents of vinylboronate were increased slightly and helped to reduce the formation of 11, the palladium loading was reduced to 1 mol %, and the overall concentration maximised at 0.66 M, without compromising the yield. The amount of water was chosen so that the potassium carbonate formed an approximately saturated solution, which could be stirred easily (smaller amounts caused the carbonate to form a sticky lump which jammed the stirrer). To simplify the workup, the cooled reaction mixture was diluted with either diethyl ether or dioxane, filtered (to remove inorganic salts), concentrated carefully under reduced pressure on a rotary evaporator, and purified by distillation up a Vigreux column (b.p. 73° C./26 mbar). Vinylpyrimidine (3) was thereby obtained in 77% yield (containing ca. 10% w/w dioxane), which would probably have been higher had compound 3 been less volatile.

We speculated whether dioxane could be replaced by THE (more volatile, less toxic, entry 9), but conversion to 3, although clean, was slow and incomplete. One consequence of being forced to use potassium vinyltrifluoroborate (in conjunction with only 2 equivalents of potassium carbonate) was the observation of significant exothermic behaviour and carbon dioxide evolution, but both were manageable on 2 mole scale.

The intriguing observation of the conjugate addition product (10) led us to try the reaction of the ammonium ylide derived from t-butyl bromoacetate and DABCO with compound 3 in refluxing acetonitrile (Scheme 4).

### Example 3. Cyclopropanation of 4-methyl-2-vinylpyrimidine (3)

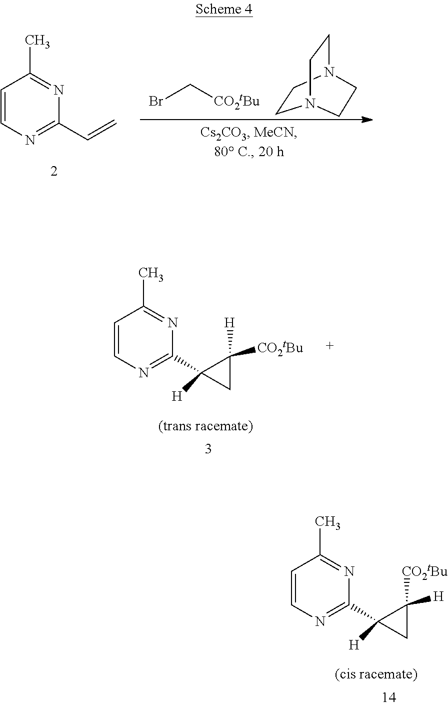

DABCO and t-butyl bromoacetate were stirred in anhydrous acetonitrile for 40 min at room temperature to form a quaternary ammonium salt. Vinylpyrimidine and cesium carbonate were added and the mixture was heated under reflux. Trial reactions showed the reaction gave the desired cyclopropane (3) in high purity, but did not proceed to completion, even with 2 equivalents of the ammonium ylide, and it did not respond to the addition of more reagents. The problem was readily solved by grinding the cesium carbonate to <150 μm and drying it. In this way, the cyclopropanation was complete after being heated overnight, and compound 3 was obtained in 86% yield as an oil after aqueous workup. The ¹H NMR of the crude product showed the presence of a pyrimidine-containing by-product, suspected to be the related cis-cyclopropane (14) (ratio 3:14=50:1). A pure sample of trans cyclopropane 3 was obtained by column chromatography and the stereochemistry was confirmed by a NOESY NMR experiment.

Gaunt et al. Angew. Chem. Int. Ed. 2003, 42, 828-831; Angew. Chem. Int. Ed. 2006, 45, 6024-6028. and Guo et al. Org. Lett., 2017, 19, 6494-6497 describe enantioselective cyclopropanations which employ chiral, quinuclidine-based alternatives to DABCO. Attempts of these cyclopropanations on 2 with t-butyl bromoacetate and catalytic O-methylquinine, DHQD-PHAL and (R)-(−)-3-hydroxyquinuclidine (Chart 1) in acetonitrile at refluxwere unsuccessful. Although the presence of the requisite N-alkyl ammonium salts was observed by LCMS, no cyclopropane was formed in any of the reactions.

### Chart 1

### Potential Chiral Catalysts for the Cyclopropanation of 3

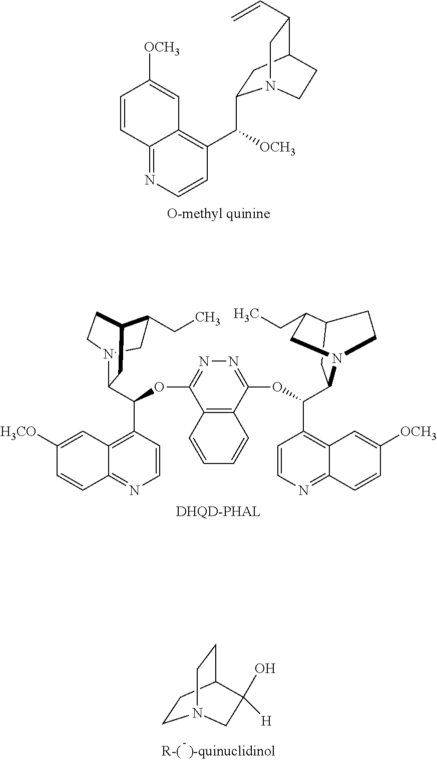

### Example 4. Preparation of (rac-trans)-2-(4-methylpyrimidin-2-yl) cyclopropane-1-carboxylic acid (rac-4)

Since 3 was an oil but had been obtained in good yield and purity, we elected to convert it to the carboxylic acid rac-4, which is crystalline, without purification. The removal of the t-butyl ester in a mixture of trifluoroacetic acid and dichloromethane was straightforward.

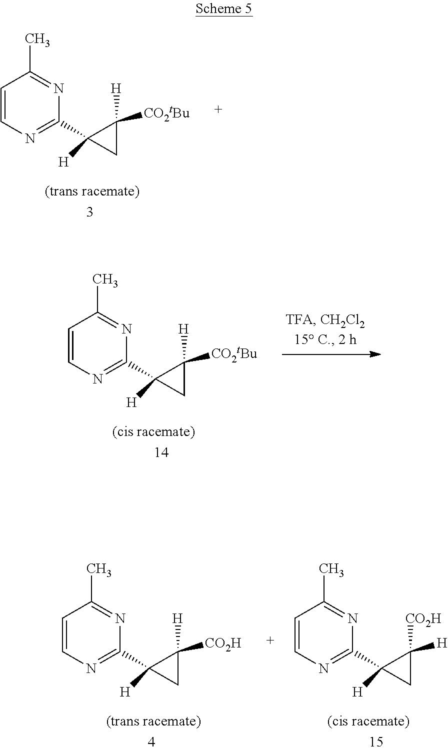 [SMILES: Cc1ccnc([C@H]2C[C@@H]2C(=O)OC(C)(C)C)n1 ; Cc1ccnc([C@H]2C[C@@H]2C(=O)OC(C)(C)C)n1 ; Cc1ccnc([C@H]2C[C@@H]2C(=O)O)n1 ; Cc1ccnc([C@H]2C[C@H]2C(=O)O)n1]

However, the isolation of the racemic acid 4 proved to be more challenging. After removal of the trifluoroacetic acid, the residue (pH 1) was freely soluble in water, and when the pH was adjusted to 4-5 (paying little attention to the overall volume of water present), multiple extractions with either dichloromethane or ethyl acetate were required, and continuous extraction with dichloromethane was necessary to obtain a good recovery. Addition of 2M aqueous sodium hydroxide to the crude trifluoroacetate salt of rac-4 led to the formation of crystals, which could be readily recovered by filtration, but the yield was variable. The pKas of rac-4 were measured and found to be 2.66 for the pyrimidine and 4.21 for the carboxylic acid. Consequently, even though the acid began to crystallize at around pH 2, the optimal condition for recovery would be at the isoelectric point of pH=3.44. The final procedure for the workup consisted of removal of the trifluoroacetic acid and dichloromethane under reduced pressure followed by slow addition of aqueous sodium hydroxide with cooling to give ˜pH 3.5. The acid was then collected by filtration and washed with water.

Analysis of the acid by ¹H NMR, isolated by crystallization from aqueous solution, showed no sign of rac-15. The LCMS trace of the acid, isolated by solvent extraction and removal of much of the trans isomer by recrystallization, revealed the presence of two peaks (RT=0.82 min (minor) and 1.94 min (major), both with m/z=179 [M+H]⁺), the latter corresponding to the desired trans isomer and the former to the evidently more polar cis isomer. Without wishing to be bound by theory, the crystallization procedure may have removed the cis isomer because it was much more polar and formed a small proportion of the mixture. A sample of pure compound rac-15 was isolated from the mother liquors of a recrystallization of rac-4, followed by reversed-phase chromatography. The structure was confirmed by 1H NMR and high resolution mass spectrometry.

Thus, this procedure is the final step of a three step preparation of the trans racemic acid 4, free from its cis isomer, without resorting to chromatography, in 58% overall yield.

### (rac-cis)-2-(4-methylpyrimidin-2-yl) cyclopropane-1-carboxylic acid (rac-cis-15)

An analytical sample was recovered from a mixture of several small-scale trial reactions after the final deprotection step. Instead of carrying out the precipitation as described for rac-4, the crude product was extracted with DCM to recover a mixture of rac-4 and rac-15. Recrystallization from EtOAc was used to remove much of the trans isomer, and the cis isomer was readily isolated using preparative reversed phase chromatography to give 20 mg of a colorless solid, after being freeze-dried. UPLCMS [Method B] RT=0.82 min; m/z 179 [M+H]⁺: purity: 100%. HRMS: Calculated for C₉H₁₁N₂O₂ (M+H)⁺: 179.0821; found 179.0814. ¹H NMR (396 MHz, CD₃OD, ppm) δ 8.50 (d. J 5.2 Hz, 1H), 7.19 (d, J 5.2 Hz. 1H), 2.77 (q, J 8.2 Hz, 1H), 2.49 (s, 3H), 2.20 (dt, J 6.6, 8.2 Hz, 1H), 1.88 (ddd, J 4.4, 6.6, 8.2 Hz, 1H), 1.48 (dt, J 4.4, 8.2 Hz, 1H). ¹³C NMR (396 MHz, CD₃OD, ppm) δ 174.9, 168.9, 167.5, 157.5, 120.0, 28.0, 23.7, 22.8, 13.2.

### Example 5. Chiral Resolution

Eleven bases were screened. (+)-Dehydroabietylamine 16 (Cheng, et. al., J. Med. Chem., 2011, 54, 957-969), (R)-and(S)-1-(2-naphthyl)ethylamine ((R)-17 and(S)-17) and(S)-1-(1-naphthyl)ethylamine ((S)-18) (Chart 2) with acid 4 gave four crystalline salts. Each of these salts was then recrystallized from a selection of solvents (Table 2) and the enrichment of the desired (1S,2S)-4 enantiomer was determined by chiral analytical SFC. The other seven, cinchonidine, quinine. O-methylquinine. (R)-(+)-α-methylbenzylamine. (S)-prolinamide, (S)-arginine, and(S)-phenylglycinol did not provide crystalline salts.

### Chart 2

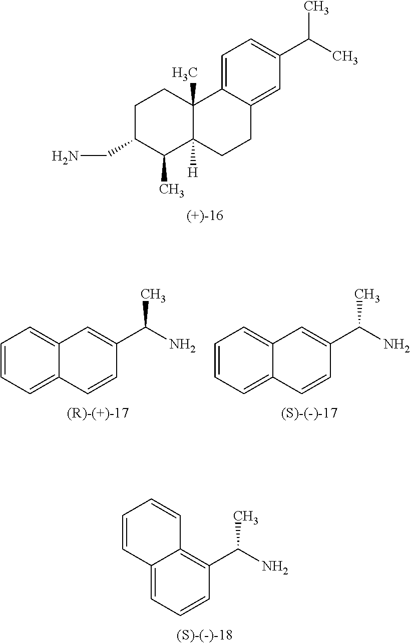

TABLE 2
Solvent screening for the resolution of carboxylic acid (4).^{a}
(+)-16 (R)-(+)-17 (S)-(−)-17 (S)-(−)-18

| | | Enantiomeric ratio of acid, (1S,2S)-(4):(1R,2R)-(4) with the amine | | | |
|---|---|---|---|---|---|
| Entry | Solvent | indicated | | | |
| 1 | Ethyl acetate | 58:42 | 22:78 | 83:16^{a} | 83:17^{b} |
| | | | | 94:6^{b} | 93:7^{c} |
| | | | | | 97:3^{d} |
| | | | | | 98.8:1.2^{e} |
| 2 | Isopropyl | 49:51 | 46:54 | 64:36 | 95:5^{f} |
| | acetate | | | | |
| 3 | Acetonitrile | insoluble | 45:54 | | |
| 4 | Acetone | 9:91 | 60:40 | | |
| 5 | Isopropanol | 68:32 | 22:78 | 81:19 | |
| 6 | THF | 3:97 | 29:71 | | |
| 7 | Toluene | 56:44 | 55:45 | 56:44 | |
| 8 | IMS | 88:12 | | | |
| 9 | MeOH | 60:40 | Very soluble | Very soluble | Very soluble |
| 10 | MeOH/H₂O = | 29:71 | | | |
| | 9:1 | | | | |
| 11 | Dioxane | | | | 94:6^{f} |
| 12 | DME | | | | 96:4^{f} |
| 13 | 2-MeTHF | | | 60:40 | 86:14 |
| 14 | Dimethyl | | | 73:27 | 92:8^{f} |
| | carbonate | | | | |

^{a}results of qualitative experiments with 100-250 mg of the rac-4 salts;
^{b}first recrystallization;
^{c}second recrystallization;
^{d}third recrystallization;
^{e}fourth recrystallization;
^{f}starting from 86:14 mixture.

Recrystallization of the (+)-dehydroabietylamine salt (16, first column of data) from THF (entry 6) was very selective but unfortunately the undesired acid formed the less soluble salt, and the antipode of dehydroabietylamine is not available commercially. Noting that recrystallization from IMS (entry 8) gave a diastereomeric salt ratio in favor of the (18,2S)-acid 4, a resolution was developed, based on a) removal of most of the unwanted diastereomeric salt by crystallization from THF (3.4 mL/mmol (rac-4)) followed by b) a single recrystallization from hot IMS (4 mL/mmol enriched salt) with controlled heating and cooling to 0° C. This procedure afforded the (1S,2S)-4-dehydroabietylamine salt (de=100%) in 23% yield (46% of theory) on a scale of 3 g. However, recrystallization of and breaking the dehydroabietylamine salt were technically more difficult than either 1-(1-naphthyl)ethylamine salt, and (+)-dehydroabietylamine is relatively more expensive, has higher molecular weight and the best grade available has only 90% purity.

A solvent screen with (R)-(+)-1-(2-naphthyl)ethylamine (rac-4 and (R)-(+)-17, second column of data) gave poor results, with ethyl acetate (entry 1) and isopropanol (entry 5) giving a diastereomeric ratio of slightly better than 3:1 in favor of the unwanted acid ((1R,2R)-acid diastereomer). A switch to(S)-(−)-1-(2-naphthyl)ethylamine (rac-4 and(S)-(−)-17, third column of data) gave predominantly the (1S,2S)-acid diastereomer in the ratio 94:6 (entry 1), after two recrystallizations from ethyl acetate, but the mass recovery was modest.

We therefore turned our attention to the(S)-(−)-1-(1-naphthyl)ethylamine salt (rac-4 and(S)-(−)-18, fourth column of data), which recrystallized nicely from ethyl and isopropyl acetate (entries 1 and 2) to afford the desired (1S,2S)-acid diastereomeric salt. Three recrystallizations from ethyl acetate (28 mL/g) were required to raise the de to 98%. However, upon examination of the mother liquors by SFC and LCMS, a peak corresponding to an acetamide derivative of the amine (19) was discovered. Although compound 19 did not contaminate the desired acid salt and remained in the mother liquors, any 19 formed would reduce the yield of the salt, and the problem would only increase with scale, as the length of the recrystallization procedure would also inevitably increase. Thus, alternative solvents of similar polarity were examined (entries 11-14). Of these, the ethers dioxane. DME and 2-MeTHF gave satisfactory results in terms of enhancement of dr, but the first two are not ideal owing to their toxicity, and 2-MeTHF gave poor crystal quality

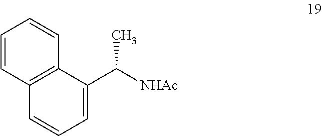 [SMILES: CC(=O)N[C@@H](C)c1cccc2ccccc12]

Trial recrystallizations of the diastereomeric mixture of salts with dimethyl carbonate revealed that 17 mL/g (5.8 mL/mmol) was important for complete dissolution at reflux, and that crystallization of the salt occurred very rapidly on cooling to around 75° C. (41% mass recovery). Another trial using the PolyBLOCK (12 mL/g, 100 to 18° C. over several hours, starting dr=86:14) gave 60% mass recovery and dr=99:1. Further refinement of the procedure demonstrated that only two recrystallizations were needed to obtain a single diastereomeric salt. An optimized procedure consisted of formation of the salt in dimethyl carbonate (0.75 mL/mmol rac-4), and after cooling, collection of the solid (dr=94.6:5.4), which was then triturated twice with more hot dimethyl carbonate (3.4 mL/mmol and 1.7 mL/mmol) to raise the dr to 99.8:0.2. The yield was 41% (82% of theory) on ca. 50 g scale. The procedure for breaking the salt consisted of stirring it with an excess of aqueous sodium hydroxide, extracting the(S)-(−)-(1-naphthyl)ethylamine into MTBE, acidifying the aqueous solution to pH 3.5 with concentrated hydrochloric acid and collecting the crystallized free acid ((1S,2S)-4, 95% yield) by filtration.

### Exemplary Salt Synthesis Using (+)-16 (1S,2S)-2-(4-methylpyrimidin-2-yl) cyclopropane-1-carboxylic acid, (1S,2S)-4, (via (+)-dehydroabietylamine salt)

(rac-trans)-2-(4-Methylpyrimidin-2-yl) cyclopropane-1-carboxylic acid (rac-4) (8.53 g, 42.6 mmol) and (+)-dehydroabietylamine (90% pure, 15.3 g, 48.1 mmol) were suspended in THF (144 mL) and the mixture was heated under reflux until a clear solution had formed (about 45 min). The solution was allowed to cool to room temperature and then placed in the fridge (8° C.) overnight. The solids were filtered and washed with cold THF (3×25 mL, −20° C.) and dried to obtain a colorless solid (11.36 g, containing 8% THF. 53%), which was a 11:89 mixture of diastereomeric salts derived from the (1S,2S)-and (1R,2R)-acid enantiomers, respectively. The mother liquors were concentrated under reduced pressure to obtain a brown solid (13.66 g, estimated to contain 9.29 g salt, 47%, dr=87:13. (1S,2S)-4: (1R,2R)-4). A 3 g portion of the solid (containing ca. 2.04 g diastereomeric salt) was suspended in IMS (12 mL) and placed in a PolyBLOCK instrument pre-heated to 85° C. and stirred until dissolved (about 30-45 min). The vial was cooled to 0° C. with the following gradient: 85-60° C. (at −0.2° C./min, over 2 h), hold at 60° C. for 1 h, then cool at −0.35° C./min. The resulting solid was collected by filtration, washed with cold IMS (−20° C. 4×5 mL) and dried under vacuum. Thus was obtained (1S,2S)-2-(4-methylpyrimidin-2-yl) cyclopropane-1-carboxylic acid-(+)-dehydroabietylamine salt (1.50 g, 74% yield adjusted for residual EtOH. 7% w/w). Chiral SFC: RT=4.55 min. ee=100%. 1H-NMR (396 MHz, CD₃OD, ppm) δ 8.40 (d, J 5 Hz. 1H), 7.16 (d, J 8 Hz. 1H). 7.10 (d, J 5 Hz, 1H), 6.96 (dd, J 8, 2 Hz, 1H), 6.87 (d, J 2 Hz, 1H), 2.94-2.75 (m, 4H), 2.59 (ddd, J 9.5, 4 Hz, 1H), 2.41-2.35 (m, 1H), 2.11 (ddd, J 8, 5, 4 Hz, 1H), 1.92-1.73 (m, 4H), 1.58-1.32 (m, 6H), 1.24 (s, 3H), 1.19 (d, J 7 Hz, 6H), 1.07 (S, 3H).

### Breaking the Dehydroabietylamine Salt

(1S,2S)-2-(4-Methylpyrimidin-2-yl) cyclopropane-1-carboxylic acid-(+)-dehydroabietylamine salt (3.76 g, 8.10 mmol) was suspended in DCM (20 mL) under nitrogen at room temperature, and aq. NaOH (6 M, 2.05 mL, 12.2 mmol) was added. The mixture was stirred for 45 min and transferred to a separating funnel with water (2 mL). The organic layer was separated, and the aqueous layer was washed with DCM (2×10 mL). The aqueous layer was treated with conc. HCl to give pH 4. The precipitated solid was collected by filtration and washed with water (2×5 mL) to give (1S,2S)-2-(4-methylpyrimidin-2-yl) cyclopropane-1-carboxylic acid, (1S,2S)-4, (1.41 g, 98%). Chiral SFC: RT=4.62 min, ee=100%.

While we have described a number of embodiments of this disclosure, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

What is claimed is:

1. A process for the preparation of a trans racemate of formula (I) 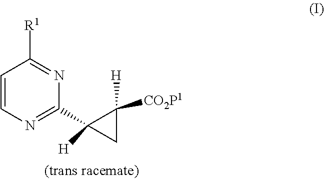
wherein
P¹ is a protecting group (such as tert-butyl)
R¹ is H or optionally substituted C₁-C₆ alkyl,
said process comprising reacting a compound of formula (II): 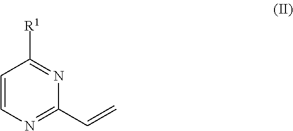
wherein R¹ is defined above for compounds of formula (I)
with a nitrogen ylid, optionally formed in situ, prepared by
reacting an α halo ester, (such as methyl chloroacetate and tert-butyl bromoacetate)
with a tertiary amine, (such as DABCO),
to form a quaternary ammonium salt,
followed by treatment with an alkali metal base (such as Cs₂CO₃ or K₂CO₃) and/or an organic base (such as DBU),
in a polar aprotic solvent (such as acetonitrile)
at an elevated temperature, (such as 70-80° C.)
such that the compound of formula (I) is formed (for example with high diastereoselectivity, such as 50:1, over the corresponding cis racemate)

2. A process according to claim 1, wherein the α halo ester is tert-butyl bromoacetate.

3. A process according to claim 1 or 2, wherein the tertiary amine is DABCO.

4. A process according to any preceding claim wherein the alkali metal base is Cs₂CO₃.

5. A process according to any preceding claim, wherein the polar aprotic solvent is acetonitrile.

6. A process according to any preceding claim wherein the elevated temperature is 70-80° C.

7. A process according to any preceding claim, wherein the reaction is performed for 12 to 30 hours, such as 13, 14, 15, 16, 17, 18, 19, 20, 21, 22, 23, 24, 25, 26, 27, 28, 29 or 30, such as 20 hours.

8. A process according to claim 1, wherein the steps, reagents and conditions are: 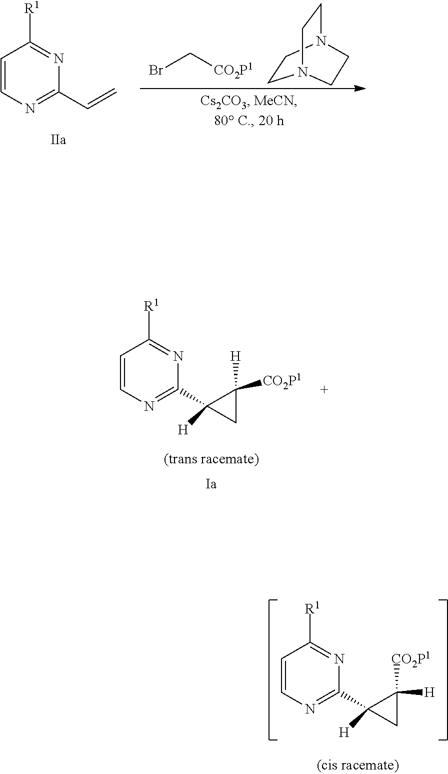
wherein R¹ and P¹ are defined above for compounds of formula (I).

9. A process according to any preceding claim 1, wherein a compound of formula (II) is prepared by reacting an intermediate of formula (III) 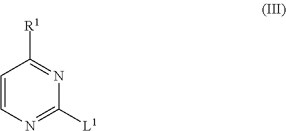
wherein R¹ is defined above for compounds of formula (I) and L¹ represents a leaving group, such as a halogen, in particular Cl
with a vinyltrihaloborate (such as vinyltrifluoroborate, in particular a salt thereof such as potassium).

10. A process according to any preceding claim wherein the protecting group P¹ is removed from compounds of formula (I) to liberate the free carboxylic acid of formula (IV): 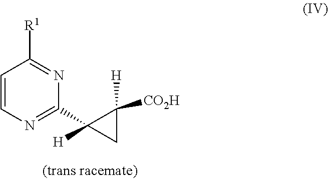
as a trans racemate (for example in high diastereomeric purity, in particular that is essentially free of the cis racemate) by:
a) acidolysis using an organic acid (such as TFA) in a chlorinated solvent (such as DCM); or
b) saponification using an alkali metal hydroxide (such as sodium hydroxide) in an aqueous medium (such THF and water),
followed by acidification with (for example aqueous hydrochloric acid), to liberate the free acid from the salt so formed.

11. A process for the resolution of a compound of formula (IV) into essentially one or other of its enantiomeric forms, for example to provide a compound of formula (V) (such as absolute as drawn): 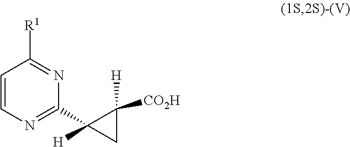
for example wherein the resolution is effected by reaction with an optically pure chiral amine (such as(S)-1-(naphthalen-2-yl) ethanamine or(S)-1-(naphthalen-1-yl) ethanamine),
in a suitable solvent (such as ethyl acetate, isopropanol and dimethyl carbonate) to form a mixture of diastereomeric salts from which the preferred diastereomeric salt crystallises and is collected by filtration.

12. A process according to claim 11, wherein the free acid is recovered from said salt by treating the latter with an excess of an aqueous solution of an alkali metal hydroxide (such sodium hydroxide or potassium hydroxide),
in the presence of an immiscible organic solvent (such as toluene or MTBE) to remove the organic base, and retain the aqueous solution.

13. A process according to claim 12, wherein the aqueous solution is acidified to pH 3-4 with an inorganic acid (such as hydrochloric acid) to precipitate the free acid as a solid which is collected by filtration.

14. A process according to any one of claims 11 to 13, wherein the compound of formula (V) is enantiomerically enriched and in particular having an enantiomeric purity (ee value) of 90% or more, such 91, 92, 93, 94, 95, 96, 96, 97, 98, 99 or 100%, especially 99%.

15. A process according to any preceding claim wherein R¹ is C₁₋₃ alkyl, such as methyl, ethyl, propyl or isopropyl, in particular methyl.

16. A process according to any preceding claim wherein P1 is C₁₋₄ alkyl such as t-butyl.

17. A process according to any one of claims 11 to 16, wherein a compound of formula (V) is reacted with an aryl amine of formula (VI): 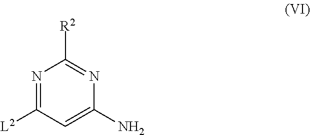
wherein R² is H, or C₁₋₃ alkyl and L², is a leaving group such as a halogen, in particular Cl, to provide a compound of formula (VII):

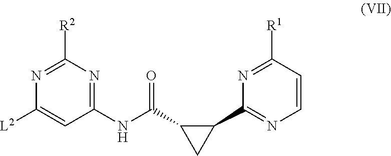

18. A process according to claim 17, wherein the compound of formula (VII) is compound (VIIa):

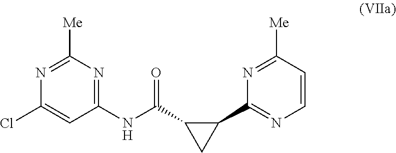 [SMILES: Cc1ccnc([C@H]2C[C@@H]2C(=O)Nc2cc(Cl)nc(C)n2)n1]

19. A process according to claim 17, wherein the compound of formula (VII) is compound (VIIb):

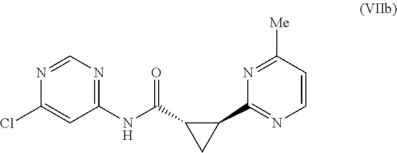 [SMILES: Cc1ccnc([C@H]2C[C@@H]2C(=O)Nc2cc(Cl)ncn2)n1]

20. A process wherein the compound of formula (VII) is reacted with a compound of formula (VIII) 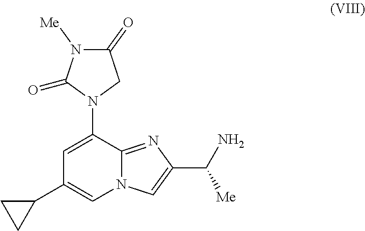 [SMILES: C[C@@H](N)c1cn2cc(C3CC3)cc(N3CC(=O)N(C)C3=O)c2n1]
to provide a compound of formula (IX) 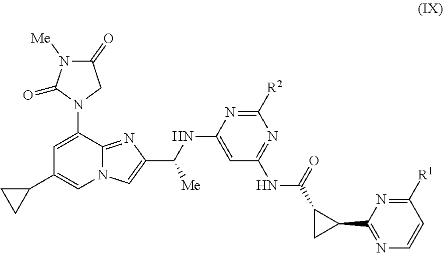
or a pharmaceutically acceptable salt thereof, wherein R¹ and R² are defined above.

21. A process according to claim 20, wherein the compound of formula (VII) is compound (VIIa) and the compound of formula (IX) is compound (IXa):

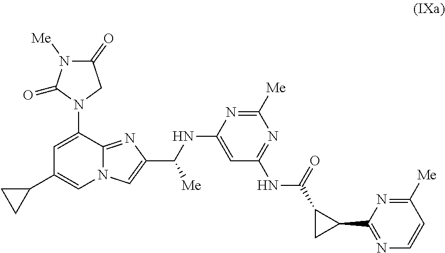 [SMILES: Cc1ccnc([C@H]2C[C@@H]2C(=O)Nc2cc(N[C@H](C)c3cn4cc(C5CC5)cc(N5CC(=O)N(C)C5=O)c4n3)nc(C)n2)n1]

22. A process according to claim 20, wherein the compound of formula (VII) is compound (VIIb) and the compound of formula (IX) is compound (IXb):

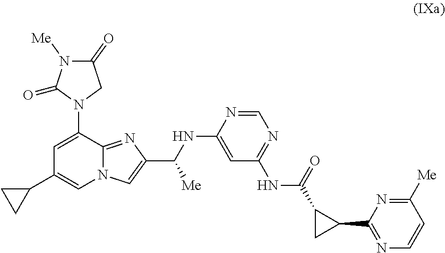 [SMILES: Cc1ccnc([C@H]2C[C@@H]2C(=O)Nc2cc(N[C@H](C)c3cn4cc(C5CC5)cc(N5CC(=O)N(C)C5=O)c4n3)ncn2)n1]

23. A compound obtained or obtainable from any one of the preceding claims.

24. A compound of formula (I), (II), (IV), (V), (VI), (VII), (VIIa) (VIII), (IX), (IXa) or (LXb).

25. A pharmaceutical composition comprising a compound according to claim 22 or 23 and an excipient, diluent or carrier.

26. A compound according to claim 23 or 24 or a pharmaceutical composition according to claim 25, for use in treatment, particularly as a pKAL inhibitor.
